(19) United States
(12) Patent Application Publication
(10) Pub. No.: US 2026/0048008 A1
(43) Pub. Date: Feb. 19, 2026

(54) ORGANOGELS FOR SUSTAINED DRUG DELIVERY, METHODS OF PREPARATION AND USE THEREOF

(71) Applicant: OCULAR THERAPEUTIX, INC., Bedford, MA (US)

(72) Inventors: Peter JARRETT, Bedford, MA (US); Rami EL-HAYEK, Bedford, MA (US); Jacob DEXTER, Bedford, MA (US); Steven LU, Bedford, MA (US); Lokendra BENGANI, Bedford, MA (US)

(21) Appl. No.: 19/102,954

(22) PCT Filed: Aug. 8, 2023
(86) PCT No.: PCT/US2023/029717

§ 371 Date: Feb. 11, 2025

Related U.S. Application Data
(60) Provisional application No. 63/397,625, filed on Aug. 12, 2022.

(51) Int. Cl. A61K 9/06; A61K 45/06; A61K 47/34

(52) U.S. Cl. CPC A61K 9/06; A61K 45/06; A61K 47/34

(57) ABSTRACT

In certain embodiments, the present invention relates to a sustained release, biodegradable drug-delivery system, comprising an organogel and an active agent, the organogel comprising a hydrophobic organic liquid, and a biodegradable, covalently crosslinked polymeric network, wherein the hydrophobic organic liquid and the active agent are contained in the biodegradable, covalently crosslinked polymeric network. In other embodiments the present invention relates to a pharmaceutically acceptable biodegradable drug-delivery system such as an implant for controlled release of a therapeutically or diagnostically active agent and methods of manufacturing it. The present invention also relates to corresponding methods of treatment and uses, as well as a kit.

### TECHNICAL FIELD

In certain embodiments, the present invention relates to a sustained release, biodegradable drug-delivery system, comprising an organogel and an active agent, the organogel comprising a hydrophobic organic liquid, and a biodegradable, covalently crosslinked polymeric network, wherein the hydrophobic organic liquid and the active agent are contained in the biodegradable, covalently crosslinked polymeric network. In particular, the present invention relates in certain embodiments to a pharmaceutically acceptable biodegradable drug-delivery system such as an implant for controlled release of a therapeutically or diagnostically active agent and methods of manufacturing it. The present invention also relates in certain embodiments to corresponding methods of treatment and uses, as well as a kit.

### BACKGROUND

Although organogels (or oleogels) have been known for decades, biomedical interest for organogels has only developed more recently. Organogels are constituted by a continuous liquid phase, typically a solvent or oil, included in a three-dimensional network. Organogels allow entrapping a vast variety of therapeutic compounds, which makes them useful as drug delivery platforms. To be considered for pharmaceutical applications, organogels need to be biocompatible.

Controlled delivery of therapeutic agents is a large area of research in the recent years. A controlled delivery improves therapies, facilitates administration and leads to better compliance, less side effects and better therapeutic results.

Another method of drug release control is to alter the chemical composition of the drug by making a pro-drug, with a different solubility that converts back into the parent drug after release from the hydrogel. This method provides viable control of release kinetics, but requires extra synthetic steps and associated testing requirements. In addition, a slow rate of pro-drug conversion to the parent drug may cause unwanted effects when released to the tissue.

Sustained delivery of hydrophilic drug compounds from a hydrogel-based implant or insert is often too fast or too slow for the desired treatment duration. This is because the rate of drug release from a water-based hydrogel increases as water solubility increases. It is desired to remove the solubility limitation. There is accordingly a need to provide a drug-delivery system allowing to control the release of active agents independent of their solubility in physiologic fluids.

### SUMMARY OF INVENTION

It is an object of certain embodiments of the invention, and an aspect, to provide a pharmaceutically acceptable, biodegradable drug-delivery system such as an implant for sustained release of an active ingredient to the body of a patient.

It is a further object of certain embodiments of the invention, and an aspect, to provide a method for manufacturing such a biodegradable drug-delivery system.

It is a further object of certain embodiments of the invention, and an aspect, to provide a method for controlling the release of an active agent from a sustained release, biodegradable drug-delivery system.

It is a further object of certain embodiments of the invention, and an aspect, to provide methods for treating a disease/medical condition of a patient with a sustained release, biodegradable drug-delivery system.

It is a further object of certain embodiments of the invention, and an aspect, to provide a kit comprising one or more sustained release biodegradable drug-delivery systems.

Some aspects of the present disclosure are directed to a sustained release, biodegradable drug-delivery system, comprising an organogel and an active agent, the organogel comprising a hydrophobic organic liquid, and a biodegradable, covalently crosslinked polymeric network, wherein the hydrophobic organic liquid and the active agent are contained, e.g., immobilized, in the biodegradable, covalently crosslinked polymeric network.

In some aspects of the present disclosure, the active agent is dissolved or dispersed in the hydrophobic organic liquid within the organogel. In other aspects, the active agent is identical to the hydrophobic organic liquid or forms at least a part thereof, i.e., the hydrophobic organic liquid consists of, consists essentially of, or comprises the active agent and optionally one or more pharmaceutically acceptable excipients.

In some aspects of the present disclosure the biodegradable, covalently crosslinked polymeric network comprises one or more polymer units of polyethylene glycol, polyethylene oxide, polypropylene oxide, polyvinyl alcohol, poly (vinylpyrrolidinone), polylactic acid (PLA), polyglycolic acid (PGA), polylactic-co-glycolic acid (PLGA), p-dioxanone, trimethylene carbonate, caprolactone, random or block copolymers and/or combinations or mixtures of any of these, or one or more units of polyaminoacids, glycosaminoglycans, polysaccharides, or proteins or combinations or mixtures of any of these.

In some aspects of the present disclosure the biodegradable, covalently crosslinked polymeric network comprises a plurality of hydrophobic polymer units such as polylactic acid (PLA), polypropylene glycol units, or polyglycolic acid (PGA) and polylactic-co-glycolic acid (PLGA) units, and/or hydrophilic polymer units such as polyethylene glycol units, polyvinyl alcohol, poly (vinylpyrrolidinone), polyethyleneimine. In certain embodiments the hydrophobic polymer units comprise polyethylene glycol units.

In some aspects of the present disclosure the polymeric network is covalently crosslinked by hydrolysable bonds between polymer units.

In some aspects of the present disclosure the polymeric network is formed from at least two multi-arm precursors (e.g., 2 to 10 arm precursors) comprising a first multi-arm precursor comprising a first functional group, and a second multi-arm precursor comprising a second functional group, the functional groups being located at the terminus of the arms, In certain embodiments, each of the first functional group and the second functional group is selected from a group consisting of an electrophile and a nucleophile, and the reaction between the first functional group and second functional group is an electrophile-nucleophile reaction that forms the covalent bond. In certain embodiments, the so-formed crosslinking covalent bond is hydrolysable under physiological conditions.

In some aspects of the present disclosure the active agent is selected from at least one of a therapeutically active agent or a diagnostically active agent or combinations thereof.

In some aspects of the present disclosure the selection of the hydrophobic liquid, and/or the hydrophobicity of the polymeric network, and/or the lactide to glycolide mole ratio (L/G ratio) is used to tune the release rate.

In some aspects of the present disclosure a release of a therapeutically effective amount of the active agent is provided for a period of time, such as up to 1 year, up to 9 months, up to 6 months, up to 3 months, up to 1 month, or up to about 25 days after administration. In other embodiments, the period of time is at least about 25 days, at least one month, at least 3 months, at least 6 months, at least 9 months or at least 1 year. In other embodiments, the period of time is from any of about 25 days, about 1 month, about 3 months, about 6 months, about 9 months or about 12 months to any of about 12 months, about 9 months, about 6 months, about 3 months, about 1 month or about 25 days. In other aspects of the present disclosure a release of a therapeutically effective amount of the active agent is provided for a period of time, such as up to three weeks, up to 2 weeks, up to 10 days, up to 9, 8, 7, 6, 5, 4, or 3 days, or up to about 1 day after administration.

In some aspects of the present disclosure the organogel delays the release of a water soluble active agent, or accelerates the release of a hydrophobic active agent.

In some aspects of the present disclosure the organogel comprises about 1 to about 90 wt.-% of the hydrophobic organic liquid; about 5 to about 95 wt.-% of the covalently crosslinked polymeric network; about 1 to about 50 wt.-% of the active agent; wherein all weight percentages are selected to amount to 100% in total, the wt.-% is based on the total weight of the drug-delivery system finished product.

Some aspects of the present disclosure are directed to a method of manufacturing a sustained release, biodegradable drug-delivery system according to any of the preceding claims, the method comprising the steps of forming an organogel from at least a covalently crosslinked polymeric network, a hydrophobic organic liquid, optionally a solvent, and at least one active agent, wherein the hydrophobic organic liquid and the active agent are contained in the biodegradable, covalently crosslinked polymeric network; and shaping the organogel as a separate step or as part of the forming step; and optionally removing solvent from the organogel.

In some aspects of the present disclosure the hydrophobic liquid is selected to adjust the hydrophobicity of the polymeric network and/or to provide a sustained release of the active agent.

In some aspects of the present disclosure the step of shaping the organogel comprises molding or extruding the reaction mixture prior to complete gelling of the organogel, allowing the mixture to gel, and optionally removing the solvent.

Some aspects of the present disclosure are directed to a sustained release, biodegradable drug-delivery system for coating a medical implant or for use as a medical implant.

In some aspects of the present disclosure the drug-delivery system is used for administration via diverse routes such as oral, parenteral (e.g., subcutaneous or intramuscular), or by operative insertion or injection.

Some aspects of the present disclosure are directed to a sustained release, biodegradable drug-delivery system for use as a medicament.

Some aspects of the present disclosure are directed to a sustained release, biodegradable drug-delivery system for use in treating a disease/medical condition of a patient, the method comprising forming an organogel comprising a therapeutically active agent dispersed in a hydrophobic organic liquid that is contained in a covalently crosslinked polymeric network, wherein the organogel is formed in situ at a treatment site of the patient, or is prefabricated and delivered to or implanted at a treatment site of the patient in order to release the active agent over an extended period of time.

Some aspects of the present disclosure are directed to a method for treating a disease/medical condition of a patient, the method comprising forming an organogel comprising a therapeutically active agent dispersed in a hydrophobic organic liquid that is contained in a covalently crosslinked polymeric network, wherein the organogel is formed in situ at a treatment site of the patient, or is prefabricated and delivered to or implanted at a treatment site in order to release the active agent over an extended period of time.

Some aspects of the present disclosure are directed to a method for treating a disease/medical condition of a patient, the method comprising administering an organogel comprising a therapeutically active agent dispersed in a hydrophobic organic liquid that is contained in a covalently crosslinked polymeric network to the patient in order to release the active agent over an extended period of time.

Some aspects of the present disclosure are directed to a method for controlling the release of an active agent from a sustained release, biodegradable drug-delivery system, by selecting a combination of a hydrophobic organic liquid (e.g., an oil) and an active agent dispersed therein, wherein either one or a combination of the following criteria applies:
a) the active agent dispersed in the hydrophobic liquid is released from the organogel together with the hydrophobic organic liquid (e.g., oil);
b) the active agent is eluted from the oil into the body directly.

Some aspects of the present disclosure are directed to a method for controlling the release of an active agent from a sustained release, biodegradable drug-delivery system, by either one or a combination (in any order or with two or more steps concurrent) of the following measures:
a) selecting the L/G ratio of the polylactic-co-glycolic acid (PLGA) units to adjust the hydrophobicity of the polymeric network;
b) selecting the L/G ratio of the polylactic-co-glycolic acid (PLGA) units to provide a sustained release of the active agent as defined herein;
c) selecting the molar ratio of the amounts of the first to second crosslinkable precursors to adjust the hydrophobicity of the polymeric network;
d) selecting the molar ratio of the amounts of the first to second crosslinkable precursors to provide a sustained release of the active agent as defined herein;
e) selecting the type of hydrophobic liquid to be contained in the organogel;
f) adding a third crosslinkable precursor that is less hydrolysable than the first and second, optionally varying the molar ratios of the three precursors
g) dispersing an active agent that has high water solubility in particulate form into the hydrophobic phase.

Some aspects of the present disclosure are directed to a kit comprising one or more sustained release biodegradable drug-delivery systems or parts thereof as described herein and instructions for using the system, and/or to a kit wherein the parts of the drug-delivery system are distributed over more than one separate containers for forming an organogel in-situ at a site of application or treatment site.

### Definitions

The term “sustained release, biodegradable drug-delivery system” as used herein refers to an object that contains an active agent and that is administered, e.g., as an implant, to a patient's body where it remains for a certain period of time while it releases the active agent into the surrounding environment. A drug-delivery system can be of any predetermined shape (e.g., rod, spherical, oblate, ellipsoidal, disc, tube, hemispherical, or irregularly shaped) before being inserted or administered, which shape may be maintained to a certain degree upon placing the system into the desired location, although dimensions of the system (e.g., length and/or diameter) may change after administration due to hydration and/biodegradation as further disclosed herein. The drug-delivery system can be designed to be biodegradable over the course of time (as disclosed below), and thus may thereby soften, change its shape and/or decrease in size, and in the end might be eliminated either by dissolution or disintegration.

The term “biodegradable” refers to a material or object (such as the drug-delivery system according to the present invention) which becomes degraded in vivo, i.e., when placed in the human or animal body or in vitro when immersed in an aqueous solution under physiological conditions such as pH 7.2-7.4 at 37° C. In the context of the present invention, as disclosed in detail herein below, the drug-delivery system comprising the organogel within which an active agent is contained, slowly (bio-)degrades over time once administered or deposited in the human or animal body. In certain embodiments, biodegradation takes place at least in part via ester hydrolysis in the aqueous environment of the body. Biodegradation may take place by hydrolysis or enzymatic cleavage of the covalent crosslinks/bonds between precursors, and/or within the polymer units of the precursors themselves. The drug-delivery system slowly softens and disintegrates, resulting in clearance through physiological pathways. In certain embodiments, the organogel of the present invention retains its shape over extended periods of time (e.g., about 1 month, 3 months, or 6 months, or longer). In certain embodiments, the shape is maintained due to covalent crosslinking of the polymer components forming the organogel, e.g., until the active agent or at least a major amount (e.g., at least 50%, at least 75% or at least 90%) thereof has been released.

An “organogel” in the present invention is a solid or semi-solid system forming a covalently crosslinked three-dimensional network of one or more hydrophilic or hydrophobic natural or synthetic polymers (as disclosed herein) that include a hydrophobic organic liquid as disclosed herein. Thus, in the present invention “organogels” are limited to so-called chemical organogels, wherein the intermolecular interaction between organogelator molecules is a chemical linkage (e.g., covalent bond) that is formed during gelation by chemical reactions inducing crosslinking. The “organogel” as used herein refers to a three-dimensional polymer network of at least two precursors/gelators that are covalently cross-linked with each other in the presence of a hydrophobic organic liquid and optionally an organic solvent and comprising the hydrophobic organic liquid contained within the covalently crosslinked polymer network.

The term “polymer(ic) network” describes a structure formed of polymer chains (of the same or different molecular structure and of the same or different molecular weight) that are covalently cross-linked with each other. The types of polymers suitable for the purposes of the present invention are disclosed herein below. The term “polymer(ic) network” is used interchangeably with the term “matrix”.

The term “amorphous” refers to a polymer or polymer network, which does not exhibit a melting point or crystalline structures in X-ray or electron scattering experiments.

The term “semi-crystalline” refers to a polymer or polymer network, which possesses some crystalline character, i.e., exhibits a melting point or some crystalline properties in X-ray or electron scattering experiments.

The term “precursor” or “gelator” or “component” herein refers to those molecules or compounds that are reacted with each other and that are thus connected via covalent crosslinks to form a polymer network and thus an organogel matrix. While other materials might be present in the organogel, such as active agents, hydrophobic liquids or solvents, they are not referred to as “precursors”.

The parts of the precursor molecules that are still present in a final polymer network are also called “units” herein. The “units” are thus the building blocks or constituents of a polymer network forming the organogel. For example, a polymer network suitable for use in the present invention may contain identical or different polyethylene glycol units, PLGA units or other types of polymers as further disclosed herein.

The term “release” (and accordingly the terms “released”, “releasing” etc.) as used herein refers to the provision of active agents from a drug-delivery system such as an implant of the present invention to the surrounding environment. The surrounding environment may be an in vitro or in vivo environment as described herein. In certain specific embodiments, the surrounding environment is the vitreous humor and/or ocular tissue, such as the retina and the choroid.

The term “100% release of the active agent” should be construed as from 95% to 100%. The way this controlled release is achieved is by a number of parameters that are characteristics of the drug-delivery system as disclosed herein. Each such characteristic feature of the drug-delivery system alone or in combination with each other can be responsible for the controlled release.

The term “sustained release” for the purposes of the present invention is meant to characterize products which are formulated to make an active agent available over an extended period of time, thereby allowing a reduction in dosing frequency compared to an immediate release dosage form, such as for example a solution of an active agent that is topically applied onto the eye (i.e., eye drops). Other terms that may be used herein interchangeably with “sustained release” are “extended release” or “controlled release”. Within the meaning of the invention, the term “sustained release” comprises constant active agent release, tapered active agent release, ascending active agent release as well as any combination thereof such as a constant active agent release followed by a tapered active agent release. Within the meaning of the invention, the term “tapered”, or “tapering” refers to a decrease of active agent release over time. Specifically, the term “sustained release” refers to release of an active agent from the drug-delivery system in a predetermined way and is in contrast to an immediate release like a bolus injection. The controlled release refers to the amount of the active agent release over the total number of days required for 100% release of the active agent in an aqueous solution under in-vitro physiological conditions such as at pH 7.2-7.4 and 37° C.

The term “extended period of time” as used herein refers to any period of time that would be considered by those of ordinary skill in the art as being extended with respect to treating a disease, and in particular refers to periods such as at least about 1 week, or at least about 1 month or longer, such as up to about 12 months, or any intermediate periods such as about 1 to about 6 months, about 2 to about 4 months, about 2 to about 3 months or about 3 to about 4 months or as otherwise disclosed herein.

A “zero order” release or “substantially zero order” release or “near zero order” release is defined as exhibiting a relatively straight line in a graphical representation of percent of the active agent released versus time. In certain embodiments of the present invention, substantially zero order release is defined as the amount of the active agent released which is proportional within 20% to elapsed time.

The terms “API”, “active (pharmaceutical) ingredient”, “active (pharmaceutical) agent”, “active (pharmaceutical) principle”, “(active) therapeutic agent”, “active”, and “drug” are used interchangeably herein and refer to the substance used in a finished pharmaceutical product (FPP) as well as the substance used in the preparation of such a finished pharmaceutical product, intended to furnish pharmacological activity or to otherwise have direct effect in the diagnosis, cure, mitigation, treatment or prevention of a disease, or to have direct effect in restoring, correcting or modifying physiological functions in a patient.

The active agent used according to the present invention may be an active agent for the treatment and/or prevention of a disease or disorder, or a diagnostic agent such as a marker. In an embodiment of the invention, the active agent is a low water solubility active agent (i.e., having a solubility in water of less than about 1000 g/mL or less than about 100 g/mL). In other embodiments of the invention, the active agent is a highly water-soluble active agent (i.e., having a solubility in water of greater than about 1000 g/mL or even greater than 10 mg/mL). This definition is not dependent on the agent being approved by a governmental agency.

For the purposes of the present invention, an active agent in all its possible forms, including free acid, free base, polymorphs or any pharmaceutically acceptable salts, anhydrates, hydrates, co-crystals, other solvates or derivatives, such as pro-drugs or conjugates, can be used. Whenever in this description or in the claims an active agent is referred to without further specification, even if not explicitly stated, it also refers to the active agent in the form of any such polymorphs, pharmaceutically acceptable salts, anhydrates, solvates (including hydrates) or derivatives thereof. With respect to the active agent, suitable solid forms include without limitation the pure substance form in any physical form known to the person of ordinary skill in the art. For example, the active agent may be in the form of particles. Particles can be amorphous or crystalline, or present a mixture of the two forms, and can be made of any size which could be without limitation classified as coarse, fine or ultrafine particles, the dimensions of which may be in particular visible to the naked eye or under the microscope and have shapes such as single grains and/or agglomerates. Particles may also be micronized. As used herein, the term “micronized” refers to small-size particles, in particular those of microscopic scale, which are without limitation reduced in particle size, by e.g., jet milling, jaw crushing, hammer milling, wet milling, precipitation in non-solvent, cryomilling (milling with liquid nitrogen or dry ice) and ball milling. An active agent can also be present in dissolved or dispersed state, e.g., within a solvent or in an aqueous medium, for example in the form of particles dispersed in an oil, or a compatible aqueous suspension which may optionally include further excipients such as a surfactant.

As used herein, the term “therapeutically effective” refers to the amount of active agent needed to produce a desired therapeutic result after administration. For example, in the context of the present invention, one desired therapeutic result would be the reduction of symptoms associated with DED, e.g., as measured by in vivo tests known to the person of ordinary skill in the art, such as an increase of a Schirmer's tear test score, a reduction of Staining values as measured by conjunctival lissamine green staining or corneal fluorescein staining, a reduction of the eye dryness severity and/or eye dryness frequency score on a visual analogue scale (VAS), a reduction of the Ocular Surface Disease Index and/or the Standard Patient Evaluation of Eye Dryness score as well as a reduction of the best corrected visual acuity. In one embodiment, “therapeutically effective” refers to an amount of active agent in a sustained release intracanalicular insert capable of achieving a tear fluid concentration which is equivalent in terms of therapeutic effect to a cyclosporine concentration of 0.236 μg/mL (which is considered to be required for immunomodulation, Tang-Liu and Acheampong, Clin. Pharmacokinet. 44(3), pp. 247-261)) over an extended period of time and in particular over substantially the whole remaining wearing period of the insert once said tear fluid concentration is achieved.

As used herein, the values “d10”, “d50”, “d90” and “d100” refer to a value characterizing the proportion of particles in a particle size distribution meeting a certain particle size. In a given particle size distribution, 10% of the particles present a particles size of d10 or less, 50% of the particles present a particles size of d50 or less, 90% of the particles present a particles size of d90 or less, and substantially all particles present a particles size of d100 or less. The percentages may be given by different parameters known to the person of ordinary skill in the art, e.g., the percentages may be based on volume, weight, or the number of the particles. Thus, d50 may exemplarily be the volume-based, the weight-based or the number-based median particle size. For example, a volume-based d90 of 43 μm means that 90% of the particles by volume have a particle size of 43 μm or less. In certain embodiments, the d10, d50 and d90 are volume-based values. The particle size distribution PSD can be commonly measured by methods as known to the person of ordinary skill in the art and includes sieving as well as laser diffraction methods. In certain embodiments, the PSD is measured by laser diffraction in accordance with USP <429> Light Diffraction Measurement of Particle Size. In certain embodiments, the PSD is measured by laser diffraction using a Beckman Coulter LS 13 320 based on the optical model “Fraunhofer.rf780z” with an obscuration value ranging from 7 to 9%.

The term “patient” herein includes both human and animal patients. The biodegradable drug-delivery systems according to the present invention are therefore suitable for human or veterinary medicinal applications. Generally, a “subject” is a (human or animal) individual to which a drug-delivery systems according to the present invention is administered. A “patient” is a subject in need of treatment due to a particular physiological or pathological condition. A “patient” does not necessarily have a diagnosis of the particular physiological or pathological condition prior to receiving the drug-delivery system.

The molecular weight of a polymer precursor as used for the purposes of the present invention and as disclosed herein may be determined by analytical methods known in the art. The molecular weight of polyethylene glycol may for example be determined by any method known in the art, including gel electrophoresis such as SDS-PAGE (sodium dodecyl sulphate-polyacrylamide gel electrophoresis), gel permeation chromatography (GPC), including GPC with static light scattering detectors (SLS) or dynamic light scattering (DLS), liquid chromatography (LC), as well as mass spectrometry such as matrix-assisted laser desorption/ionization-time of flight (MALDI-TOF) spectrometry or electrospray ionization (ESI) mass spectrometry. The molecular weight of a polymer, including a polyethylene glycol precursor as disclosed herein, is an average molecular weight (based on the polymer's molecular weight distribution), and may therefore be indicated by means of various average values, including the weight average molecular weight (Mw) and the number average molecular weight (Mn). In the case of the crosslinkable polymer gelators, such as polyethylene glycol, PLGA and poloxamer-based precursors as used in the present invention, the molecular weight indicated herein is the number average molecular weight (Mn) determined by gel permeation chromatography using a polystyrene standard, according to standard methods known in the art. Typically, the materials, especially the multi-arm precursors are purchased with a specified molecular weight defined by the vendor. Suitable PEG precursors are for example available from a number of suppliers, such as Jenkem Technology, Sinopeg, Sigma-Aldrich, and others.

The term “day 1” as used herein refers to a time point that immediately follows after “day 0”. Thus, whenever “day 1” is used, it refers to an already elapsed time period of one day or about 24 hours after administration of the drug-delivery system.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that.

The term “average” as used herein refers to a central or typical value in a set of data(points), which is calculated by dividing the sum of the data(points) in the set by their number (i.e., the mean value of a set of data).

As used herein, the singular forms “a,” “an,” and “the” include plural references unless the context clearly indicates otherwise.

The term “and/or” as used in a phrase such as “A and/or B” herein is intended to include both “A and B” and “A or B”.

Open terms such as “include,” “including,” “contain,” “containing” and the like mean “comprising.” These open-ended transitional phrases are used to introduce an open-ended list of elements, method steps, or the like that does not exclude additional, unrecited elements or method steps.

The term “up to” when used herein together with a certain value or number is meant to include the respective value or number.

The terms “from A to B”, “of from A to B”, and “of A to B” are used interchangeably herein and all refer to a range from A to B, including the upper and lower limits A and B.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

The abbreviation “PBS” when used herein means phosphate-buffered saline.

The abbreviation “PEG” when used herein means polyethylene glycol.

The abbreviation “PLGA” when used herein means poly(lactic-co-glycolic acid). If not specified otherwise, it has a L/G ratio of 1:1 (50:50)

The term “hydrophobic”, or “lipophilic” is defined as a property of polymers or materials having a low degree of water attraction or absorption, i.e., the material is repelled from a mass of water. The term “hydrophilic”, or “lipophobic” is vice versa defined as a property of materials or polymers that attract water or have a strong affinity for water. Hydrophobicity may be measured by determining contact angles of drops of liquids, preferably water droplets, formed on a solid polymer and/or gel surface. Furthermore, hydrophobic organic liquids as used in the present invention are immiscible or at least not readily miscible with water.

The term “immobilized” as used herein refers to long range immobility and does not refer to localized mobility within the matrix, i.e., the hydrophobic liquid phase is present as a continuous phase within the polymer matrix and may be slowly mobile only in vivo, i.e., it can slowly escape into body fluid over time.

The term “syneresis” describes the phenomenon of separating out of liquid (oil) from the (organo-)gel during contraction of the gel.

### BRIEF DESCRIPTION OF THE DRAWINGS

FIG. 1 illustrates the basic structure of Organogels versus Hydrogels.

FIG. 2 is a photograph of the organogels of Example 1.

FIG. 3 is a graph of the in vitro release of the drug-delivery systems of Examples 2A-2F.

FIG. 4 is a graph of the in vitro release of the drug-delivery systems of Examples 3A-3D.

FIG. 5 is a graph of the in vitro release of the drug-delivery systems of Examples 4A-4H.

FIG. 6 is a graph illustrating the in-vitro bupivacaine base release over time of some of the organogel formulations of Example 5.

FIG. 7 is a graph illustrating the in-vitro release over time of travoprost organogel formulations of Example 6 at different doses and temperatures.

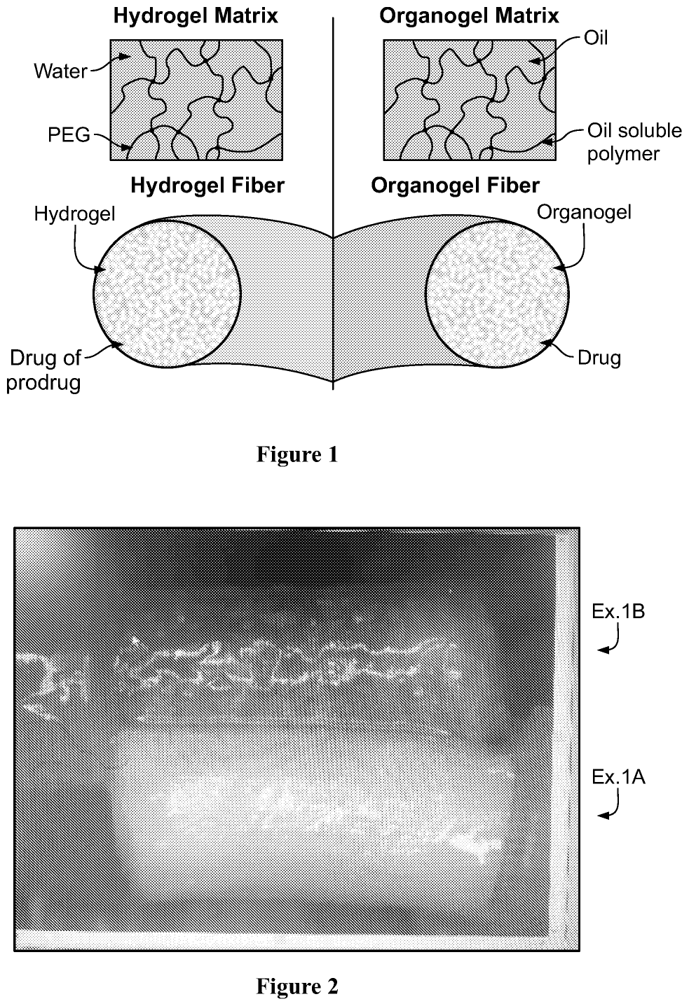

### DETAILED DESCRIPTION OF THE INVENTION

In certain embodiments, the present invention provides a sustained release, biodegradable drug-delivery system, comprising an organogel and an active agent, the organogel comprising a hydrophobic organic liquid, and a biodegradable, covalently crosslinked polymeric network, wherein the hydrophobic organic liquid and the active agent are contained in the biodegradable, covalently crosslinked polymeric network. In certain embodiments, a sustained release, biodegradable drug-delivery system is provided, comprising an organogel and an active agent, the organogel comprising a hydrophobic organic liquid, and a biodegradable, covalently crosslinked polymeric network, wherein the hydrophobic organic liquid and the active agent are immobilized in the biodegradable, covalently crosslinked polymeric network.

The sustained release, biodegradable drug-delivery system in certain embodiments comprises at least three constituents, a biodegradable covalently crosslinked polymer network, a hydrophobic organic liquid and an active agent.

The organogel in certain embodiments is formed by polymerization of non-linear, multifunctional monomeric or polymeric precursor components as disclosed herein later and forms a covalently crosslinked polymeric network that includes the hydrophobic organic liquid and immobilizes it within the polymeric network, e.g., until it is released from the network in vivo. The organogels of the present invention are thus like a hydrophobic analog to hydrogels that include water instead of a hydrophobic organic phase. Organogels are similar to hydrogels in that the matrix is composed of a network forming polymeric component (gelator) and a non-reactive component. In a hydrogel the non-reactive component is water, whereas in the organogel of the present invention it is a hydrophobic organic compound with glass (Tg) and melt (Tm) transition temperatures below body temperature, such as an oil.

Covalent crosslinking of the polymer network forming precursors in certain embodiments provides a limited mobility to the hydrophobic organic liquid (e.g., oil) component. This may provide continuous control of drug release by limiting drug transport to diffusion through the organogel and/or eliminating development of defects that provide fast escape routes for the drug from developing. In certain embodiments, the drug-delivery system of the present invention is a fully or partly diffusion controlled delivery system, i.e., the release of the oil and/or the active agent is primarily controlled by diffusion processes. Degradation of the polymer matrix may additionally occur in the organogels of the present invention, but does not primarily control the release of the active agent. In non-crosslinked gels such as extruded linear polymers the release of the active agent is mainly controlled by degradation of the polymeric matrix that releases the active agent mainly in a degradation controlled system. The network forming precursors should be miscible in the hydrophobic organic liquid component, such that when crosslinked it “holds” the component to create a solid or semi-solid, that forms the organogel. In certain embodiments, the hydrophobic organic liquid compatibility with the polymer network has an impact on the rate the hydrophobic organic liquid escapes into the surrounding tissue fluid in vivo, and may gradually be replaced by aqueous fluid, providing an additional method of controlling drug release kinetics to active agent solubility and network degradation.

In certain embodiments, the use of an organogel in the sustained release, biodegradable drug-delivery system of the invention thus allows to modify the release of an active agent from the drug-delivery system by tailoring or suitably selecting the precursor components forming the crosslinked polymeric network according to their hydrophilic and/or hydrophobic properties. Furthermore, in certain embodiments, the release of an active agent from the drug-delivery system can be modified or controlled by suitably selecting the hydrophobic organic liquid according to its properties such as hydrophobicity, viscosity, compatibility with the active agent, solubility or insolubility of the active agent in the hydrophobic organic phase, and the like.

The organogel based drug-delivery system of certain embodiments of the present invention offers several advantages over hydrogels. For example, certain organogels are anhydrous, so water degradable (hydrolysable) components such as water sensitive active agents can be stabilized and made storage stable over extended periods of time and don't require hydration at the time of implantation.

Water soluble compounds have low solubility or are insoluble in organogels, allowing the drug to be incorporated as a particulate solid embedded in the organogel matrix. The low solubility of the drug in the organogel matrix provides a mechanism to control the rate of drug release. This property vastly increases the range of compounds that can be contained in an implant.

Manipulation of the lipophilicity/hydrophilicity of the organogel can be used to adjust the release rate of a drug and to influence diffusion rates. Pure hydrogels cannot be adjusted this way, since they are water-based, thus in these systems the drug itself has to be modified to a prodrug form for such adjustment of drug/matrix solubility. In organogels, this can be avoided. Additionally, varying the lipophilicity/hydrophilicity of the organogel can be further used influence degradation rate of the polymer matrix, also having an additional influence on the release rate of a drug.

The organogel can be designed to release the hydrophobic organic liquid (e.g., oil) from the matrix slowly in vivo, allowing a slow conversion to hydrogel that is then degraded. This provides a new mode of drug release control and increases biocompatibility.

Optional addition of a solvent in organogels can be used during manufacture to overcome compatibility issues of the components, and the solvent can be removed to yield an organogel with an immobilized oil. Removal of the solvent can be accomplished by heat treatment, which is not possible for materials that undergo melting or glass transitions at elevated temperatures. Solvent removal can also be accomplished by methods typically employed for non-crosslinked polymers, such as water extraction, vacuum drying, lyophilization, evaporation, etc. Elimination of the need for careful removal of solvent greatly simplifies the fabrication process.

In certain embodiments, organogels possess the physical qualities of low modulus, dimensional stability, and favorable drug release kinetics. In certain embodiments, organogels can be dimensionally stable to heat and will not melt. Thus, implant fabrication processes such as hot melt extrusion can be used to form certain organogels of the present invention.

The drug-delivery system of the present invention comprising an organogel may be used to deliver classes of drugs including steroids, non-steroidal anti-inflammatory drugs (NSAIDS), intraocular pressure lowering drugs, antibiotics, peptides, or others. The organogel may be used to deliver drugs and therapeutic agents, e.g., an anti-inflammatory (e.g., Diclofenac), a pain reliever (e.g., Bupivacaine), a calcium channel blocker (e.g., Nifedipine), an Antibiotic (e.g., Ciprofloxacin), a cell cycle inhibitor (e.g., Simvastatin), a protein or peptide (e.g., Insulin), enzymes, antineoplastic agents, local anesthetics, hormones, angiogenic agents, anti-angiogenic agents, growth factors, antibodies, neurotransmitters, psychoactive drugs, anticancer drugs, chemotherapeutic drugs, drugs affecting reproductive organs, genes, and oligonucleotides, or other configurations, as well as viruses such as AAV for gene delivery. The rate of release from the organogel may depend on the properties of one or more of the active agent, the hydrophobic organic liquid and the polymer network, with other possible factors including one or more of drug sizes, relative hydrophobicity, organogel density, organogel solids content, and the like.

The drug-delivery system of the present invention may be in the form of an implant, a medical implant or a pharmaceutically acceptable implant, an implant coating, or an oral dosage form, etc.

### Hydrophobic Organic Liquid/Oil

The hydrophobic organic liquid can be used to modify the release of an active agent from the drug-delivery system. One or more of its properties such as hydrophobicity, viscosity, compatibility with the active agent, solubility or insolubility of the active agent in the hydrophobic organic phase, and the like can be suitably selected to control the release of the active agent form the organogel. For example, when the sustained release, biodegradable drug-delivery system of the invention is an implant inserted into the human body, or an oral dosage form, the hydrophobic organic liquid can diffuse out of the organogel into the aqueous environment together with the active agent dissolved therein, before or concurrently with a diffusion of the active agent out of the hydrophobic organic liquid. For example, the release of hydrophobic drugs may be accelerated by co-diffusion with the oil from the organogel. If the active agent is, for example, a water-soluble solid particulate dispersed in the hydrophobic organic liquid, the organic liquid can be used to slow down contact of the aqueous environment with the active agent and to delay leaching out of the active agent from the organogel.

In certain embodiments, the hydrophobic organic liquid is liquid at human body temperature, such as a temperature of about 37° C. or lower, or liquid in the range of 0° C. to 40° C., or 10° C. to 38° C., or 15° C. to 37° C., or 25° C. to 37° C., or at 37° C. The term “liquid” may include viscous fluids having a creamy or wax-like but non-solid appearance. Also, for some hydrophobic organic liquids that undergo hydration in aqueous embodiments such as body fluids the melting point at a certain temperature may be different for the hydrated material than for the non-hydrated. In certain embodiments of the present invention, the hydrated form of such materials are liquid under those conditions as described above.

In an embodiment the active agent is dissolved or dispersed in the hydrophobic organic liquid. In another embodiment, the active agent is the hydrophobic organic liquid or forms at least a part thereof.

In certain embodiments, the hydrophobic organic liquid is an oil, or comprises an oil or oil mixture. It may be a biocompatible vegetable oil, a synthetic oil or a mineral oil, a liquid fatty acid or triglyceride composition, or it may be a hydrophobic biodegradable liquid polymer, or combinations thereof.

In certain aspects of the present disclosure the hydrophobic organic liquid is a biocompatible oil that may be selected from the group comprising triethyl citrate, acetyl triethyl citrate (ATEC), acetyl tributyl citrate (ATBC), α-tocopherol (vitamin E), α-tocopherol acetate; plant or vegetable oils such as sesame oil, olive oil, soybean oil, sunflower oil, coconut oil, canola oil, rapeseed oil, nut oils such as hazelnut, walnut, pecan, almond, cottonseed oil, corn oil, safflower oil, linseed oil, etc., ethyl oleate, castor oil and derivatives thereof (Cremophor®), lipids being liquid at 37° C. or lower, such as saturated or unsaturated fatty acids, monoglycerides, diglycerides, triglycerides (Myglyols®), isopropyl myristate, phospholipids, glycerophospholipids, sphingolipids, sterols, prenols, polyketides, hydrophobic biodegradable liquid polymers (such as low molecular weight PLGA, PGA or PLA etc.), low melting point waxes such as plant, animal or synthetic waxes, lanolin, jojoba oil, or combinations thereof.

In certain aspects, the hydrophobic organic liquid is liquid at human body temperature, and may have a glass transition temperature and/or a melting temperature equal to or below 45° C., or equal to or below 37° C.

In certain embodiments, the hydrophobic organic liquid is non-volatile at 37° C. and ambient pressure, non-toxic, and/or biocompatible, and/or capable of being cleared from an implantation site, metabolized and/or cleared unchanged from the body.

### Polymer Network

The organogel of the biodegradable drug-delivery system of the invention in certain embodiments comprises a covalently crosslinked polymer network that is formed by polymerization of multifunctional precursor components. In an embodiment, at least one of the precursors has a functionality of equal to or greater than 3 in order to create a three-dimensional (3D) polymer network and is thus non-linear.

In the organogel the biodegradable, covalently crosslinked polymer network may comprise one or more polymer units comprising polyalkylene oxides such as polyethylene glycol, polypropylene glycol, poly(ethylene glycol)-block-poly(propylene glycol) copolymers, poloxamers such as Tetronic®, polyethylene oxide, polypropylene oxide; polyvinyl acetate, polyvinyl alcohol, poly (vinylpyrrolidinone), polylactic acid (PLA), polyglycolic acid (PGA), polylactic-co-glycolic acid (PLGA), p-dioxanone, trimethylene carbonate, caprolactone, random or block copolymers or combinations or mixtures of any of these, or one or more units of polyaminoacids, glycosaminoglycans, polysaccharides, or proteins, while this list is not intended to be limiting.

In an embodiment of the invention, hydrophobic polymer units may include at least one poly(ethylene glycol)-block-poly(propylene glycol) copolymer, also known as poloxamers such as commercially available Tetronic® poloxamers.

The biodegradable, covalently crosslinked polymeric network may be formed of a plurality of hydrophobic polymer units, or from a plurality of hydrophilic polymer units, or a combination of hydrophobic and hydrophilic units. The polymer units may be selected to tailor the hydrophobicity and hydrophilicity of the organogel in order to adjust it to the properties of the hydrophobic organic phase and/or the active agent. This adjustment enables control of gel formation as well as degradation behavior of the organogel.

In an embodiment of the invention, hydrophobic polymer units may include at least one of polylactic acid (PLA), and polylactic-co-glycolic acid (PLGA) units, preferably copolymers of PEG and PLGA, particularly preferred block copolymers of multiarm PEG copolymerized with PLGA. The copolymer may be end-capped with the desired reactive groups, and the molecular weight of PEG and the PEG/PLGA ratio in the copolymer may be varied according to desired hydrophobicity.

Hydrophilic polymer units may be selected from at least one of polyethylene glycol units, polypropylene glycol units, or polyglycolic acid (PGA). In one embodiment, the hydrophilic polymer units comprise polyethylene glycol units.

Each of the polymer units may have an average molecular weight (Mw) in the range from about 1,000 to about 100,000 Daltons, or from about 10,000 to about 60,000 Daltons, or from about 15,000 to about 50,000 Daltons.

In an aspect of the invention, the covalently crosslinked polymeric network comprises a combination of a plurality of hydrophobic polymer units selected from at least one of polylactic acid (PLA), and polylactic-co-glycolic acid (PLGA), and a plurality of at least one of hydrophilic polymer units selected from at least one of polyethylene glycol (PEG) units, polypropylene glycol (PPG), or polyglycolic acid (PGA) units. In one embodiment the hydrophilic polymer units comprise polyethylene glycol (PEG) units.

In an embodiment, the polymeric network comprises a combination of polylactic-co-glycolic acid (PLGA) units and polyethylene glycol (PEG) units. The ratio of polylactic-co-glycolic acid (PLGA) units to polyethylene glycol (PEG) units can be selected to be about 2.5:1 to about 1:2.5, or about 2:1 to about 1:2, or about 1:1.

When PLGA is used, the polylactic-co-glycolic acid (PLGA) units can have an L/G ratio (in % L or G units) ranging from 0:100 to 100:0, or 1:99 to 99:1, or 10:90 to 90:10, or 25:75 to 75:25, or 50:50.

In certain embodiments, in the organogel of the sustained release drug delivery system the polymeric network is covalently crosslinked by hydrolysable bonds between polymer units, which facilitates biodegradation in aqueous environments such as the human or animal body in vivo.

The hydrolysable bonds can include bonds selected from the group consisting of amine, amide, urethane, ester, anhydride, ether, acetal, ketal, nitrile, isonitrile, isothiocyanate, or imine bonds, and combinations thereof. These bonds are typically formed by condensation polymerization reactions of suitably functionalized gelators or precursors, respectively.

### Precursor Components

In certain embodiments, the polymer network of the organogel is formed from at least one covalently crosslinkable precursor that is miscible with the hydrophobic organic liquid, preferably soluble or dispersible in the hydrophobic organic liquid, or optionally a mixture of the hydrophobic liquid and a solvent.

According to certain embodiments of the invention, the organogel comprises a polymer network comprising at least two covalently crosslinked multi-arm precursors. In some embodiments, the organogel comprising a polymer network includes at least two covalently crosslinked multi-arm precursors.

Thus, a precursor is always a “functional polymer” or “functional material” such as a crosslinker (e.g., with low molecular weight) that is able to participate in the crosslinking reaction with another precursor to form a covalently crosslinked polymer network (or matrix). Thus, the term “non-functional polymer” refers to a polymer that may be present in the organogel of the present invention but does not participate in the crosslinking reaction with the precursors to form a polymer network.

The precursor used in the invention may be any polymer as long as it is able to react with another precursor in the presence of the hydrophobic organic liquid and is biocompatible. The polymer may be selected from a biodegradable natural, semisynthetic, synthetic, or biosynthetic polymer.

Natural polymers may include glycosaminoglycans, polysaccharides (e.g., dextran), polyaminoacids and proteins or mixtures or combinations thereof. Semisynthetic polymers may be selected from carboxymethyl celluloses, or alkyl celluloses such as methyl cellulose (MC), ethyl cellulose (EC).

In some aspects, synthetic precursors are utilized. Synthetic refers to a molecule not found in nature or not normally found in a human. Synthetic polymer may generally be any polymer that is synthetically produced by different types of polymerizations, including free radical polymerization, anionic or cationic polymerization, chain-growth or addition polymerization, condensation polymerization, ring-opening polymerization etc. The polymerization may be initiated by certain initiators, by light and/or heat, and may be mediated by catalysts.

Generally, for the purposes of the present invention one or more synthetic polymers of the group comprising one or more units of polyalkylene glycol, such as polyethylene glycol (PEG), polypropylene glycol, poly(ethylene glycol)-block-poly(propylene glycol) copolymers, or polyethylene oxide, polypropylene oxide, polyvinyl alcohol, poly(vinylpyrrolidinone), polylactic acid (PLA), polylactic-co-glycolic acid (PLGA), p-dioxanone, trimethylene carbonate, caprolactone; random or block copolymers or combinations/mixtures of any of these can be used, while this list is not intended to be limiting.

In some aspects of the invention, at least one crosslinkable precursor is either hydrophobic or hydrophilic, and when two precursors are used, both may be hydrophobic, or both may be hydrophilic, or one is hydrophobic and the other one is hydrophilic. With more than two precursors, any mixture of hydrophilic and hydrophobic precursors may be chosen, depending on the desired properties of the polymer network. In addition, the precursors can be copolymers, incorporating both hydrophobic and hydrophilic substructures.

The precursors have functional groups that react with each other, i.e., a first functional group capable of reacting with a second functional group. The functional groups react with each other, for example, in electrophile-nucleophile reactions or are configured to participate in other polymerization reactions. Thus, the first functional group may be a nucleophile and the second functional group may be an electrophile, or vice versa. According to certain embodiments of the invention, each precursor comprises at least two nucleophiles or at least two electrophiles.

Nucleophiles that can be used for the present invention may comprise an amine such as a primary amine, a hydroxyl, a thiol, a carboxyl, a dibenzocyclooctyne, or a hydrazide. In certain embodiments, at least one precursor comprises a nucleophile, such as a primary amine.

Electrophiles that can be used for the present invention may comprise succinimidyl esters, succinimidyl carbonates, nitrophenyl carbonates, aldehydes, ketones, acrylates, acrylamides, maleimides, vinylsulfones, iodoacetamides, alkenes, alkynes, an azide, norbornenes, epoxides, mesylates, tosylates, tresyls, cyanurates, orthopyridyl disulfides, or halides. These electrophiles comprise functional groups that participate in the electrophile-nucleophile reaction and crosslink the precursors, and they preferably additionally include reactive groups that include hydrolysable groups or bonds, such as glutarate. For example, in an embodiment of the invention, a succinimidyl ester may comprise a reactive group such as succinimidyl succinate (SS), succinimidyl glutarate (SG), succinimidyl adipate (SAP), succinimidyl azelate (SAZ), or succinimidyl glutaramide.

The term “multi-arm” precursors means that the precursors are branched, i.e., non-linear. In the case of a multi-arm polymer, a core refers to a contiguous portion of a molecule joined to arms that extend from the core, with the arms having a nucleophile or electrophile, which is often at the terminus of the branch. Precursors may have, e.g., 2-100 arms, with each arm having a terminus, bearing in mind that some precursors may be dendrimers or other highly branched materials such as dendrimers. An arm on a precursor refers to a linear chain of chemical groups that connect a cross linkable group to a polymer core. Some embodiments are precursors with between 3 and 300 arms; artisans will immediately appreciate that all the ranges and values within the explicitly stated ranges are contemplated, e.g., 4, 6, 8, 10, 12, 4 to 16, 8 to 100, 6, 8, 10, 12, or at least 4 arms.

In certain embodiments, the multi-arm precursor of the invention has a core and from 2 to 10 arms, or 3 to 10 arms, 4 to 8 arms, or 4 or 8 arms, each arm comprising a polymer unit and having a terminus.

In some embodiments, when each precursor is multi-arm, it comprises two or more arms and thus, two or more same or different electrophiles or nucleophiles, such that each nucleophile may react with another electrophile (within the same precursor or another precursor) in an electrophilic-nucleophilic reaction to form a crosslinked polymeric product. Thus, for example, in some aspects, the precursor has 4 arms, wherein each arm terminates with either a nucleophile or an electrophile that may or may not be the same as its other arms.

According to an aspect of the invention, the organogel comprises at least two multi-arm precursors comprising a first multi-arm precursor comprising nucleophiles and/or electrophiles, and a second multi-arm precursor comprising nucleophiles and/or electrophiles. In this embodiment, the first multi-arm precursor and the second multi-arm precursor are covalently cross-linked with each other in an electrophile-nucleophile reaction. In this context, the multi-arm refers to at least 4 arms, at least 8 arms, such as at least 10 arms.

In one embodiment, if the organogel comprises two multi-arm precursors, it may comprise a first multi-arm precursor comprising a nucleophile such as an amine such as a primary amine, a thiol, a dibenzocyclooctyne, or a hydrazide, and a second multi-arm precursor comprising an electrophile such as succinimidyl esters, succinimidyl carbonates, nitrophenyl carbonates, aldehydes, ketones, acrylates, acrylamides, maleimides, vinylsulfones, iodoacetamides, alkenes, alkynes, azides, norbornenes, epoxides, mesylates, tosylates, tresyls, cyanurates, orthopyridyl disulfides, or halides. The nucleophile and electrophile are covalently cross-linked to each other in an electrophile-nucleophile reaction. In some embodiments, the first multi-arm precursor is a primary amine, and the second multi-arm precursor is a succinimidyl ester.

According to certain embodiments of the invention, the organogel comprises at least two multi-arm precursors comprising a first multi-arm precursor comprising nucleophiles and/or electrophiles, and a second multi-arm precursor comprising nucleophiles and/or electrophiles. In this embodiment, the first multi-arm precursor and the second multi-arm precursor, are covalently cross-linked with each other in an electrophile-nucleophile reaction. In this context, the multi-arm refers to at least 4 arms, at least 8 arms, such as at least 10 arms.

In an embodiment, the organogel comprises at least two multi-arm precursors comprising a first multi-arm precursor comprising a nucleophile, and a second multi-arm precursor comprising an electrophile. In this embodiment, the first multi-arm precursor and the second multi-arm precursor are covalently cross-linked with each other in an electrophile-nucleophile reaction. In this context, the multi-arm refers to at least 4 arms, at least 8 arms, such as at least 10 arms. In this embodiment, the nucleophile can be an amine such as a primary amine, a thiol, a dibenzocyclooctyne, or a hydrazide, and the electrophiles can be succinimidyl esters, succinimidyl carbonates, nitrophenyl carbonates, aldehydes, ketones, acrylates, acrylamides, maleimides, vinylsulfones, iodoacetamides, alkenes, alkynes, azides, norbornenes, epoxides, mesylates, tosylates, tresyls, cyanurates, orthopyridyl disulfides, or halides. For example, in an embodiment of the invention, a succinimidyl ester may comprise a reactive group such as succinimidyl succinate (SS), succinimidyl glutarate (SG), succinimidyl adipate (SAP), succinimidyl azelate (SAZ), or succinimidyl glutaramide.

Some precursors may have a longer hydrolysis half-life as compared to others. This means that the time required for them to degrade may be longer. This may, in part, be due to the reactive group comprised in that precursor. For example, a PEG polymer comprising an electrophile group such as a succinimidyl ester group that comprises a reactive group such as a succinimidyl succinate (SS) has a shorter hydrolysis half-life as compared to a PEG polymer comprising an electrophile group such as a succinimidyl ester group that comprises a reactive group such as a succinimidyl glutarate (SG).

In an embodiment, the organogel comprises two multi-arm precursors and it may comprise a first multi-arm precursor comprising a nucleophile such as an amine, and a second multi-arm precursor comprising an electrophile such as a succinimidyl ester. In another embodiment, the organogel may comprise a first multi-arm precursor comprising a nucleophile such as an amine such as a primary amine, and a second multi-arm precursor comprising an electrophile such as a succinimidyl ester comprising a first reactive group. In this embodiment, the reactive group is selected from succinimidyl succinate (SS), succinimidyl glutarate (SG), succinimidyl adipate (SAP) or a succinimidyl azelate (SAZ).

In some embodiments, precursors are polyethylene glycol precursors. Thus, in some embodiments, the polymer network of covalently cross-linked precursors is made of or includes at least one polyethylene glycol-containing precursor. Polyethylene glycol (PEG, also referred to as polyethylene oxide) refers to a polymer with a repeat group (CH₂CH₂O)n, with n being at least 3.

A polymeric precursor having a polyethylene glycol thus has at least three of these repeat groups connected to each other in a linear series. A PEG polymer that terminates in a hydroxyl group or a methoxy group that does not participate in the cross-linking reaction between the precursors is referred to as a “non-functional PEG” described herein above and thus, not used as one of the precursors. Thus, a PEG polymer that terminates in a nucleophile selected from a primary amine, a thiol, a dibenzocyclooctyne, or a hydrazide is considered as a “functional PEG” and can be used as one of the precursors. Further, a PEG polymer that terminates in an electrophile selected from succinimidyl esters, succinimidyl carbonates, nitrophenyl carbonates, aldehydes, ketones, acrylates, acrylamides, maleimides, vinylsulfones, iodoacetamides, alkenes, alkynes, azides, norbornenes, epoxides, mesylates, tosylates, tresyls, cyanurates, orthopyridyl disulfides, or halides is considered as a “functional PEG” and can be used as one of the precursors.

The polymer network of the organogel drug-delivery systems of the present invention may comprise one or more multi-arm PEG units having from 2 to 10 arms, or 4 to 8 arms, or 4, 5, 6, 7 or 8 arms. The PEG units may have a different or the same number of arms. In certain embodiments, the PEG units used in the organogel of the present invention have 4 and/or 8 arms. In certain embodiments, a combination of 4- and 8-arm PEG units is utilized.

In certain embodiments of the present invention, polyethylene glycol units used as precursors have an average molecular weight in the range from about 1,000 to about 100,000 Daltons, or in a range from about 10,000 to about 60,000 Daltons, or in a range from about 15,000 to about 50,000 Daltons. In certain embodiments the polyethylene glycol units have an average molecular weight in a range from about 10,000 to about 40,000 Daltons, or of about 20,000 Daltons. PEG precursors of the same average molecular weight may be used, or PEG precursors of different average molecular weight may be combined with each other. The average molecular weight of the PEG precursors used in the present invention is given as the number average molecular weight (Mn), which, in certain embodiments, may be determined by gel permeation chromatography against polystyrene standard according to standardized methods.

In a 4-arm PEG, each of the arms may have an average arm length (or molecular weight) of the total molecular weight of the PEG divided by 4. A 4a20kPEG precursor, which is one precursor that can be utilized in the present invention thus has 4 arms with an average molecular weight of about 5,000 Daltons each. An 8a20k PEG precursor, which may be used in addition to the 4a20kPEG precursor in the present invention, thus has 8 arms each having an average molecular weight of 2,500 Daltons. Accordingly, a 4a20K PLGA precursor has 4 arms with an average molecular weight of about 5,000 Daltons each.

When referring to a PEG precursor having a certain average molecular weight, such as a 15kPEG- or a 20kPEG-precursor, the indicated average molecular weight (i.e., a Mn of 15,000 or 20,000, respectively) refers to the PEG part of the precursor, before end groups are added (“20k” here means 20,000 Daltons, and “15k” means 15,000 Daltons—the same abbreviation is used herein for other average molecular weights of PEG or other polymer precursors). In certain embodiments, the Mn of the PEG part of the precursor is determined by gel permeation chromatography against polystyrene standard according to standardized methods. The degree of substitution with end groups as disclosed herein may be determined by means of H-NMR after end group functionalization.

In various embodiments of the invention, the organogel comprises at least two multi-arm precursors, the first precursor is a multi-arm PEG precursor comprising a nucleophile such as an amine, such as a primary amine. In some of these embodiments, the second multi-arm precursor is a multi-arm PEG precursor comprising an electrophile such as a succinimidyl ester. In other of these embodiments, the second multi-arm precursor is a multi-arm PLGA precursor comprising an electrophile such as a succinimidyl ester

In some embodiments of the invention, the organogel comprises three multi-arm precursors, the first multi-arm precursor is a multi-arm PEG precursor comprising a nucleophile such as an amine, such as a primary amine. In this embodiment, the second multi-arm precursor is a multi-arm PEG precursor comprising an electrophile such as a succinimidyl ester comprising a first reactive group. In this embodiment, the third multi-arm precursor is a multi-arm PEG precursor comprising an electrophile such as a succinimidyl ester comprising a second reactive group. In this embodiment, the first and the second reactive groups can be selected from succinimidyl succinate (SS), succinimidyl glutarate (SG), succinimidyl adipate (SAP) or a succinimidyl azelate (SAZ). SS, SG, SAP, and SAZ are all functionalization linkers attached to the polymer comprising reactive groups composed of N-succinimidyl esters of the corresponding diacids, that have an ester group connection to the polymer at the second acid of the diacid that can degrade by hydrolysis in water. In some embodiments, the first multi-arm precursor is succinimidyl succinate (SS) and the second multi-arm precursor is succinimidyl glutarate (SG).

Each and any combination of electrophilic- and nucleophilic-group containing PEG precursors disclosed herein may be used for preparing the implant according to the present invention. For example, any 4-arm or 8-arm PEG precursor (e.g., having a succinimidyl ester comprising a SS, SG, SAP, or SAZ reactive group) may be combined with any 4-arm or 8-arm PEG precursor (e.g., having a NH₂ group or another nucleophile). Furthermore, the PEG units of the electrophile- and the nucleophile group-containing precursors may have the same or may have a different average molecular weight.

One such combination is a PEG amine precursor and two PEG succinimidyl ester precursors, one comprising an SS reactive group and another comprising an SG reactive group. In certain embodiments, the inventors have found that by keeping the molar ratio of PEG amine to PEG succinimidyl ester at about 1:1 and by varying the molar ratio of the reactive groups of the succinimidyl esters SS and SG, the time taken by the polymeric network to degrade in an aqueous solution under physiological conditions can be controlled, although other ratios are contemplated. The amount of PEG SS and SG to be used to reach a particular molar ratio of the two reactive groups can be calculated by a skilled artisan and described as follows.

The amount of PEG amine and PEG esters (SS and SG) to be used is calculated through stoichiometric equations of molar proportion and converting moles to grams. First, the reactive end group molar ratio between the amine, the succinimidyl succinate, and succinimidyl glutarate is determined. In an example formulation, 4a20k PEG NH₂, 4a20k PEG SS, and 4a40k PEG SG are used. The molar ratio between amine and succinimidyl ester groups is about 1:1, and the molar ratio between SS and SG is about 80:20. The final end group molar ratio between the 4a20k NH₂:4a20k SS:4a40k SG is about 1.0:0.8:0.2. Next, gram to mole stoichiometric conversions, and vice versa, are used to determine mass amounts. Below outlines an example calculation of 4a20k SS at the molar ratios above with 100 g of 4a20k NH₂:

$$100 g 4 a 20 k PEG NH 2 \times \frac{1 mol PEG NH 2}{20000 g PEG NH 2} \times \frac{4 mol NH 2}{1 mol PEG NH 2} \times \frac{0.8 mol SS}{1 mol NH 2} \times \frac{1 mol PEG SS}{4 mol SS} \times \frac{20000 g PEG SS}{1 mol PEG SS} = 80 g 4 a 20 k PEG SS$$

Alternatively, the amounts of PEGs can be determined by calculating the “molecular weight between crosslinks” (MWc) and the arm length ratio. The MWc can be calculated through the sum of the average arm length of each multi-arm PEG precursor.

$$PEG Arm Length = \frac{PEG MW}{\# arms} \times PEG molar ratio$$

$$MWc = PEG NH 2 Arm Length + PEG SS Arm Length + PEG SG Arm Length$$

The arm length ratio is calculated by dividing the PEG Arm Length over the MWc. By multiplying the arm length ratio for a particular multi-arm precursor with a total PEG batch size, the amount of multi-arm precursor can be determined. Below outlines an example calculation for the amount of 4a20k PEG SS with a total batch size of 100 g PEG:

$$PEG NH 2 Arm Length = \frac{20000 Da}{4} \times 1. = 5000 Da$$

$$PEG SS Arm Length = \frac{20000 Da}{4} \times 0.8 = 4000 Da$$

$$PEG SG Arm Length = \frac{40000 Da}{4} \times 0.2 = 2000 Da$$

$$MWc = 5000 Da + 4000 Da + 2000 Da = 11000 Da$$

$$PEG SS Arm Length Ratio = \frac{40000 Da}{11000 Da} = 0.364$$

$$Mass PEG SS = 0.364 \times 100 g = 36.4 g$$

Similar calculations can be done for other types of polymers as described herein.

In certain embodiments, 4-arm PEGs with an average molecular weight of about 20,000 Daltons and 4-arm PEGs with an average molecular weight of about 40,000 Daltons can be used for forming the polymer network and thus the organogel according to the present invention.

Thus, the first precursor, and/or the second precursor may be a 4a20k precursor, wherein 4 denotes the arms and 20k denotes the Mn. Thus, for example, the first, second and/or the third precursor may be a 4a40k precursor. Thus, for example, the first and/or the second precursor may be a 4a20k precursor and the third precursor may be a 4a40k precursor.

If the polymer unit is PLGA instead of PEG, such precursors may have the following exemplary structure with a pentaerythritol derived core of a rather hydrophobic and oil soluble 4a20K PLGA-NHS:

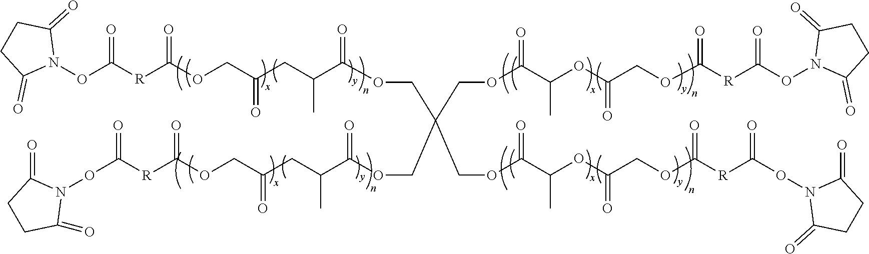

According to its designation, this is a 4 arm PLGA with each PLGA unit having a Mn of about 5,000 Daltons, and the PLGA units have an L/G ratio of 50:50 (i.e., 1:1), R together with the two carbonyl groups to which it is bound is part of a diacid linker derived from a saturated or unsaturated biocompatible organic diacid, such as one of oxalic acid, malonic acid, succinic acid, glutaric acid, adipic acid, maleic acid, fumaric acid, and NHS designates a N-hydroxy succinimide electrophile as the functional group on each arm's terminus. x is an integer and define the number of lactic acid units and y is an integer defining the number of glycolic acid units in the PLGA molecule. For 50:50 PLGA x and y are equal. n is an integer defining the number of PLGA blocks, for 50:50 PLGA n is 1.

Another example of an electrophile functionalized PLGA precursor is 4a20K PLGA5050-SAP-NHS (x and y are about 15):

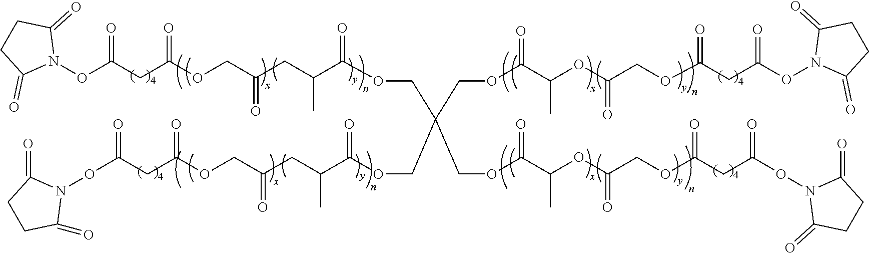

In other embodiments, the multi-arm PLGA precursor may also be derived from ethylenediamine as the core instead of pentaerythritol.

In various embodiments of the invention, the organogel comprises at least one multi-arm precursor that comprises hydrophobic polymer units selected from polylactic acid (PLA) units and polylactic-co-glycolic acid (PLGA) units, or combinations or (block)copolymers thereof. In such embodiments the organogel includes at least one crosslinker, preferably a small molecule amine such as tris(2-aminoethyl)amine (TAEA), which is oil soluble, or trilysine.

In various embodiments of the invention, the organogel comprises at least one multi-arm precursor that comprises hydrophobic polymer units selected from polylactic acid (PLA) units and polylactic-co-glycolic acid (PLGA) units, or combinations or (block)copolymers thereof, and at least one further multi-arm precursor comprising hydrophilic polymer units, preferably selected from polyethylene glycol (PEG) and polyglycolic acid (PGA).

As described above, the polymeric network is formed from at least two precursors, at least one of them being a multi-arm precursor, and a first multi-arm precursor comprising a first functional group, and a second precursor selected from a small molecule crosslinker or a multi-arm precursor comprising a second functional group, the functional groups being located at the terminus of the arms or the molecule. In various embodiments of the invention, each of the first functional group and the second functional group is selected from a group consisting of an electrophile and a nucleophile, and the reaction between the first functional group and second functional group is an electrophile-nucleophile reaction that forms the covalent bonds in the polymer network.

The nucleophile and the electrophile are selected from the groups as defined herein before. In certain embodiments, the nucleophile is an amine group, and the electrophile is an activated ester group.

### Active Agents:

The active agent according to the invention can be a therapeutically active agent or a diagnostically active agent, or combinations thereof. It may be a single active agent or a plurality of active agents.

For the purposes of the present invention, an active agent includes all its possible forms, including free acid, free base, polymorphs, pharmaceutically acceptable salts, anhydrates, hydrates, other solvates, stereoisomers, crystalline forms, cocrystals, pro-drugs, conjugates (e.g., pegylated compounds), complexes and mixtures thereof. For the purpose of the present invention, all forms of the active agent are intended to be pharmaceutically acceptable. The term “salt,” as used herein, can include, but is not limited to, inorganic acid salts such as hydrochloride, hydrobromide, hydroiodite, sulfate, phosphate and the like; organic acid salts such as formate, acetate, trifluoroacetate, maleate, tartrate, glutarate and the like; sulfonates such as methanesulfonate, benzenesulfonate, p-toluenesulfonate, and the like; and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like. Any salt used herein is meant to be a pharmaceutically acceptable salt. The term “co-crystal” as used herein refers to a combination of an active pharmaceutical ingredient (API) and one or more co-formers, such as acids (such as carboxylic acids) in the same lattice through non-covalent interactions, such as hydrogen bonds, electrostatic interactions, π-π stacking, van der Waals interactions, etc. Co-crystals are thus multi-component solids. The difference between co-crystals and salts is that the former are only composed of neutral components, while the latter contain ionic components. Cocrystallization may alter, and in certain cases and for certain applications optimize, the physicochemical properties of an API, for example regarding stability, solubility, dissolution rate, mechanical properties etc.

Therapeutically active agents used herein may be immunosuppressants, complement inhibitors (e.g., CS inhibitors such as eculizumab or avacincaptad pegol), steroids, anti-inflammatories such as steroidal and non-steroidal anti-inflammatories (e.g., COXI or COX 2 inhibitors), antivirals, antibiotics, anti-glaucoma agents, anti-VEGF agents, analgesics, tyrosine kinase inhibitors, integrin inhibitors, IL-6 blockers, reactive aldehyde species (RASP) inhibitors, nitric oxide donating PgAs, antihistamines, mast cell stabilizers, rho kinase inhibitors, plasma kallikrein inhibitors, BCL-2 blockers, semaphorin antagonists, HtRA I blockers, IGF-1 R inhibitors, VEGF combination agents (multi-specific antiangiogenic agents) and combinations thereof.

Therapeutically active agents may be steroids; non-steroidal anti-inflammatory drugs (NSAIDS) such as Diclofenac, Ibuprofen, Meclofenamate, Mefanamic A, Salsalate, Sulindac, Tolmetin, Ketoprofen, Diflunisal, Piroxicam, Naproxen, Etodolac, Flurbiprofen, Fenoprofen C, Indomethacin, Celecoxib, Ketorolac, Nepafenac; intraocular pressure lowering drugs; antibiotics such as Ciprofloxacin; pain reliever such as Bupivacaine; calcium channel blockers such as Nifedipine; cell cycle inhibitors such as Simvastatin; proteins such as insulin; small molecule hydrophilic drugs, including carboxylic acid salts and amine salts; small molecule hydrophobic drugs, hydrophilic peptides and protein drugs, such as insulin, single chain antibody fragments, Fab fragments, IgG antibodies, fusion antibodies, etc.; aptamers; particularly Bupivacaine (BPV-HCl or base), Ropivacaine (RPV), Dexamethasone, Travoprost, Axitinib, non-steroidal anti-inflammatory drugs (NSAIDS), steroids, antibiotics, pain relievers, calcium-channel blockers, cell cycle inhibitors, chemotherapeutics, anti-viral drugs, anesthetics, hormones, anticancer drugs, antineoplastic agents, etc., or any combinations thereof.

In some embodiments, steroids may be corticosteroids that can comprise hydrocortisone, loteprednol, cortisol, cortisone, prednisolone, methylprednisolone, dexamethasone, betamethasone, triamcinolone, aldosterone, fludrocortisone, budesonide, fluocinolone, mometasone, fluticasone, rimexolone, fluorometholone, beclomethasone, or flunisolide,

In some embodiments, NSAIDs can comprise at least one of diclofenac (e.g., diclofenac sodium), flurbiprofen (e.g., flurbiprofen sodium), ketorolac (e.g., ketorolac tromethamine), bromfenac, nepafenac, cyclooxygenase-1 (COX-I) and cyclooxygenase-2 (COX-2), isozymes, salicylates, propionic acid derivatives, acetic acid derivatives, enolic acid derivatives, and anthranilic acid derivatives. acetylsalicylic acid, diflunisal, salsalate, ibuprofen, dex-ibuprofen, naproxen, fenoprofen, ketoprofen, dex-ketoprofen, oxaprozin, loxoprofen, indomethacin, tolmetin, sulindac, etodolac, aceclofenac, nabumetone, piroxicam, tenoxicam, lornoxicam, phenylbutazone, mefenamic acid, meclofenamic acid, flufenamic acid, tolfenamic acid, celecoxib, pharmaceutically acceptable salts thereof and combinations thereof.

In some embodiments, the active agent may be an analgesic selected from at least one of acetaminophen, acetaminosalol, aminochlorthenoxazin, acetylsalicylic 2-amino-4-picoline acid, acetylsalicylsalicylic acid, anileridine, benoxaprofen, benzylmorprune, 5-bromosalicylic acetate acid, bucetin, buprenorphine, butorphanol, capsaicin, cinchophen, ciramadol, clometacin, clonixin, codeine, desomorphine, dezocine, dihydrocodeine, dihydromorprune, dimepheptanol, dipyrocetyl, eptazocine, ethoxazene, ethylmorphine, eugenol, floctaferune, fosfosal, glafenine, hydrocodone, hydromorphone, hydroxypethidine, ibufenac, p-lactophenetide, levorphanol, meptazinol, metazocine, metopon, morprune, nalbuphine, nicomorphine, norlevorphanol, normorphine, oxycodone, oxymorphone, pentazocine, phenazocine, phenocoll, phenoperidine, phenylbutazone, phenylsalicylate, phenylramidol, salicin, salicylamide, tiorphan, tramadol, diacerein, actarit, pharmaceutically acceptable salts thereof and combinations thereof.

In some embodiments, IOP lowering agents and/or glaucoma medications can comprise prostaglandin analogs (e.g., bimatoprost, latanoprost, travoprost, or latanoprostene bunod), rho kinase inhibitor (e.g., netarsudil), adrenergic agonists (epinephrine or dipivefrin), beta-adrenergic antagonists also known as beta blockers (e.g., timolol, levobunolol, metipranolol, carteolol, or betaxolol), alpha2-adrenergic agonists (e.g., apraclonidine, brimonidine, or brimonidine tartrate), carbonic anhydrase inhibitors (e.g., brinzolamide, dichlorphenamide, methazolamide acetazolamide, acetazolamide, or dorzolamide), pilocarpine, echothiophate, demercarium, physostigmine, and/or isofluorophate.

In some embodiments, anti-infective can comprise antibiotics comprising ciprofloxacin, tobramycin, erythromycin, ofloxacin, gentamicin, fluoroquinolone antibiotics, moxifloxacin, and/or gatifloxacin, aminoglycosides, penicillins, cephalosporins, fluoroquinolones, macrolides, and combinations thereof. Aminoglycosides may include tobramycin, kanamycin A, amikacin dibekacin, gentamicin, sisomicin, netilmicin, neomycin B, neomycin C, neomycin E, streptomycin, paromomycin, pharmaceutically acceptable salts thereof and combinations thereof. Penicillins may include:

Amoxicillin, ampicillin, bacampicillin, carbenicillin, cloxacillin, dicloxacillin, flucloxacillin, mezlocillin, nafcillin, oxacillin, penicillin G, penicillin V, piperacillin, pivampicillin, pivmecillinam, ticarcillin, pharmaceutically acceptable salts thereof and combinations thereof. Cephalosporins may include: cefacetrile, cefadroxil, cefalexin, cefaloglycin, cefalonium, cefaloridine, cefalotin, cefapiriu, cefatrizine, cefazaflur, cefazedone, cefazolin, cefradine, cefroxadine, ceftezole, cefaclor, cefamandole, cefrnetazole, cefonicid, cefotetan, cefoxitin, cefprozil, cefuroxime, cefuzonam, cefcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, cefoperazone, ceftazidime, cefclidine, cefepime, cefluprenam, cefoselis, cefozopran, cefpirome, cefquinome, ceftobiprole, ceftaroline, cefaclomezine, cefaloram, cefaparole, cefcanel, cefedrolor, cefempidone, cefetrizole, cefivitril, cefmatilen, cefinepidium, cefovecin, cefoxazole, cefrotil, cefsumide, cefuracetime, ceftioxide, pharmaceutically acceptable salts thereof and combinations thereof. Fluoroquinolones may include ciprofloxacin, levofloxacin, gatifloxacin, moxifloxacin, ofloxacin, norfloxacin, pharmaceutically acceptable salts thereof and combinations thereof. Macrolides may include azithromycin, erythromycin, clarithromycin, dirithromycin, oxithromycin, telithromycin, pharmaceutically acceptable salts thereof and combinations thereof.

In some embodiments, the active agent may be selected from antivirals comprising nucleoside reverse transcriptase inhibitors, non-nucleoside reverse transcriptase inhibitors, fusion inhibitors, integrase inhibitors, nucleoside analogs, protease inhibitors, and reverse transcriptase inhibitors. Examples of antiviral agents include, but are not limited to: abacavir, aciclovir, acyclovir, adefovir, amantadine, amprenavir, ampligen, arbidol, atazanavir, boceprevir, cidofovir, darunavir, delavirdine, didanosine, docosanol, edoxudine, efavirenz, emtricitabine, enfovirtide, eutecavir, famciclovir, fomivirsen, fosamprenavir, foscarnet, fosfonet, ganciclovir, ibacitabine, imunovir, idoxuridine, imiquimod, iodinavir, inosine, interferon type III, interferon type II, interferon type I, interferon, lamivudine, lopinavir, loviride, maraviroc, moroxydine, methisazone, nelfiuavir, nevirapine, nexavir, oseltamivir, peginterferon alfa-2a, penciclovir, peramivir, pleconaril, podophyllotoxin, raltegravir, ribavirin, rimantadine, ritonavir, pyramiding saquinavir, stavudine, tenofovir, tenofovir disoproxil, tiprauavir, trifluridine, trizivir, tromantadine, truvada, valaciclovir, valganciclovir, vicriviroc, vidarabine, viramidine, zalcitabine, zanamivir, zidovudine, pharmaceutically acceptable salts thereof and combinations thereof. In certain embodiments the antiviral is one of ganciclovir, idoxuridine, vidarabine, and/or trifluridine.

In some embodiments, the active agent may be selected from antifungals comprising amphotericin B, natamycin, voriconazole, fluconazole, miconazole, clotrimazole, ketoconazole, posaconazole, echinocandin, caspofungin, and/or micafungin.

In some embodiments, antimetabolites can comprise methotrexate, mycophenolate, or azathioprine.

In some embodiments, antifibrotic agents can comprise mitomycin C or 5-fluorouracil.

In some embodiments, angiogenesis inhibitors can comprise anti-VEGF agents (e.g., aflibercept, ranibizumab, bevacizumab, brolucizumab, conbercept), PDGF-B inhibitors (e.g., Fovista®), complement antagonists (e.g., eculizumab), tyrosine kinase inhibitors (e.g., axitinib, deucravacitinib, avapritinib, capmatiuib, pegimatiuib, ripretinib, selpercatinib, selumetinib, tucatinib, entrectinib, erdaftinib, fodratiuib, pexidartiuib, upadacatinib, zanubrutinib, baricitinib, biuimetinib, dacomitinib, fostamatinib, gilteritinib, larotrectinib, lorlatinib, acalabrutiuib, brigatinib, midostaurin, neratinib, alectiuib, cobimetiuib, lenvatinib, osimertinjb, ceritinib, nintedanib, afatinib, ibrntinib, trametinib, bosutinib, cabo antinib, ponatinib, regorafenib, lofacitiuib, crizotinib, ruxolitiuib, vandetanib, pazopanib, lapatinib, nilotinib, dasatiuib, sunitinib (vorolanib), sorafenib, erlotinib, gefitinib, imatinib, afatinib, bosutinib, cabozantinib, cediranib, ceritinib, crizotinib, dabrafenib, dasatinib, erlotinib, everolimus, gefitinib, imatinib, lestaurtinib, nilotinib, palbociclib, pazopanib, ponatinib, regorafenib, ruxolitinib, semananib, sirolimus, sorafenib, temsirolimus, tofacitinjb, trametinib, vandetanib, and vemurafenib), and/or integrin antagonists (e.g., natalizumab and vedolizumab). In another embodiment, the tyrosine kinase inhibitor can be a Src family tyrosine kinase inhibitor, such as but not limited to: A419259, AP23451, AP23464, AP23485, AP23588, AZD0424, AZM475271, BMS354825, CGP77675, CU201, ENMD 2076, KB SRC 4, KX2361, KX2-391, MLR 1023, MNS, PCI-32765, PD166285, PD180970, PKC-412, PKI166, PP1, PP2, SRN 004, SU6656, TC-S7003, TG100435, TG100948, TX-1123, VAL 201, WH-4-023, XL 228, altenusin, bosutinib, damnacanthal, dasatinib, herbimycin A, indirubin, neratinib, lavendustin A, pelitinib, piceatannol, saracatinib, Srcll, foretinib, motesanib, tivozanib, LY2457546, MGCD-265, MGCD-510, tivantinib, AMG458, JNJ-3887, EMD1214063, BMS794833, PH11665752, SGX-523, lNCB280, pharmaceutically acceptable salts thereof and combinations thereof.

In some embodiments, the active agent may be an immunosuppressant selected from at least one of cyclosporine, mTOR inhibitors (e.g., rapamycin, tacrolimus, temsirolimus, sirolimus, everolimus, KU-0063794, WYE-354, AZD8055 metformin, or Torin-2), cyclophosphamide, etoposide, thiotepa, methotrexate, azathioprine, mercaptopurine, interferons, infliximab, etanercept, mycophenolate mofetil, 15-deoxyspergualin, thalidomide, glatiramer, leflunomide, vincristine, cytarabine, pharmaceutically acceptable salts thereof and combinations thereof. Active agents may also be selected from anti-inflammatory-cytokine targeting agents such as Target TNFa, IL-I, IL-4, IL-5, IL-6, or IL-17, or CD20. Such agents may include etanercept, infliximab, adalimumab, daclizumab, rituximab, tocilizumab, certolizumab pegol, golimumab, pharmaceutically acceptable salts thereof and combinations thereof.

In some embodiments, for example for ophthalmic drug delivery systems, active agents may be selected from anti-glaucoma agents including beta-blockers such as atenolol propranolol, metipranolol, betaxolol, carteolol levobetaxolol, levobunolol timolol, pharmaceutically acceptable salts thereof and combinations thereof, adrenergic agonists or sympathomimetic agents such as epinephrine, dipivefrin, clonidine, apraclonidine, brimonidine, pharmaceutically acceptable salts thereof and combinations thereof, parasympathomimetic or cholinergic agonists such as pilocarpine, carbachol, phospholine iodine, physostigmine, pharmaceutically acceptable salts thereof and combinations thereof; carbonic anhydrase inhibitor agents, including topical or systemic agents such as acetozolamide, brinzolamide, dorzolamide; methazolamide, ethoxzolamide, dichlorphenamide, pharmaceutically acceptable salts thereof and combinations thereof; mydriatic-cycloplegic agents such as atropine, cyclopentolate, succinylcholine, homatropine, phenylephrine, scopolamine, tropicamide, pharmaceutically acceptable salts thereof and combinations thereof; prostaglandins such as prostaglandin F2 alpha, antiprostaglandins, prostaglandin precursors, or prostaglandin analog agents such as bimatoprost, latanoprost, travoprost, unoprostone, tafluprost, pharmaceutically acceptable salts thereof and combinations thereof.

In some embodiments, cytoprotective agents can comprise ebselen, sulforaphane, oltipraz or dimethyl fumarate.

In some embodiments, neuroprotective agents can comprise ursodiol, memantine or acetylcysteine.

In some embodiments, anaesthetic agents can comprise lidocaine, proparacaine or bupivacaine.

In some embodiments, the active agent can be dexamethasone, ketorolac, diclofenac, vancomycin, moxifloxacin, gatifloxicin, besifloxacin, travoprost, 5-fluorouracil, methotrexate, mitomycin C, prednisolone, bevacizumab (Avastin®), ranibizumab (Lucentis®), sunitinib, pegaptanib (Macugen®), timolol, latanoprost, brimonidine, nepafenac, bromfenac, triamcinolone, difluprednate, fluocinolide, aflibercept, or combinations thereof. In some embodiments, the agent may be dexamethasone, ketorolac, diclofenac, moxifloxacin, travoprost, 5-fluorouracil, or methotrexate. In some embodiments, the agent is dexamethasone. In some embodiments, the agent is ketorolac. In some embodiments, the agent is travoprost.

In some embodiments, the active agent may be selected from at least one of cyclosporine, everolimus, tacrolimus, sirolimus, pimecrolimus, ibuprofen, mefanamic acid, diclofenac, nepafenac, flurbiprofen, flurbiprofen sodium, fusidic acid, besifloxacin (base), clarithromycin, azithromycin, ketotifen (base), azelastine (base), azelastine embonate, linoleic acid, alpha-linolenic acid, gamma-linolenic acid, prednisone, prednisolone, prednisolone acetate, methylprednisolone, dexamethasone, dexamethasone acetate, betamethasone sodium phosphate, budesonide, flunisolide, fluticasone propionate, triamcinolone, triamcinolone acetonide, triamcinolone hexacetonide, triamcinolone diacetate, fluocinolone acetonide, fludrocortisone acetate, loteprednol, loteprednol etabonate, difluprednate, fluorometholone, mometasone furoate, deoxycorticosterone acetate, aldosterone, rimexolone, beclometasone, beclomethasone dipropionate and lifitegrast.

In some embodiments, the active agent can be selected from at least one of peptides, nanobodies, affibody molecules, ankyrins and DARPins. Peptides may be Compstatin, APL-I, Fc-III-4C, Beovu (Brolucizumab), Zimura (Avacincaptad Pegol), Pegcetacoplan, Abicipar Pegol, Larnpalizumab, Fovista, Risuteganib, AXT107, Elamipretide, THR149, ALM201, VGB3, and Largazole. Nanobodies may be selected from GaNOTA-Anti-HER2-VHH1, GaNOTA-Anti-HER2-VHH1, mTc-NM-02, 131I-SGMIB-Anti-HER2-VHH1, GaNOTA-Anti-MMR-VHH2, mTc-Anti-PD-L1, L-DOS47+Doxorubicin, L-DOS47+Cisplatin/Vinorelbine, KN035+Trastuzumab/Docetaxel, KN035, KN044, TC-210 T Cells, CD19/CD20 bispecific CART cells, BCMA CART cells, or TAS266 nanobodies. Affibody molecules may be those described in Stahl et al., Affibody Molecules in Biotechnological and Medical Applications, Trends in biotechnology 2017, 35 (8) p. 691-712, which is incorporated herein by reference in its entirety. Ankyrins and DARPins are described, for example, in a review by Caputi et al., Current Opinion in Pharmacology 2020, 51:93-101, which is incorporated herein by reference in its entirety. MP0250, a tri-specific DARPin drug candidate that can bind VEGF-A and hepatocyte growth factor (HGF) as well as one molecule of MP0250 binding two molecules of human serum albumin (HSA); Abicipar pegol (MPO112 or AGN-150998); Brolucizumab, Ranibizumab, or Aflibercept.

In some embodiments, the therapeutically active agent can be selected from at least one of complement inhibitors including those that target: Cl/Cl Q, CJ, CJ Convertase, CS, CS convertase, C5a, C5aR, C6, C7, C8, C9, CD59, Factor B, Factor D, Factor H, Factor P, or combination thereof. These may include particular agents such as cinryze, berinert, ruconest, sutimlimab, pegcetacoplan (GA), eculizumab, ravuilizumab, avacopan, pozelimab, nomacopan, zilucopan, vilobelimab, crovalimab, avacincapted pegol), cemdisiran, BDB-001, tesidolumab, avdoralimab, MOR210, ALXN1720, danicopan, vemircopan, ACH-5228, ACH-5548, BCX-9330, AMY-101, ANX005, ANX007, narsoplimab, iptacopan, CLG561, GT103, ARGX-117, ALXN1820, NGM621, lampalizumab, NGM621, IONIS-FB-Lrx, GEM I 03, CLG561, pharmaceutically acceptable salts thereof combinations thereof.

In some embodiments, the therapeutically active agent can be selected from at least one of antihistamines such as loratadine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimipramine doxylamine, pheniramine, pyrilamine, chiorcyclizine, thonzylamine, pharmaceutically acceptable salts thereof and combinations thereof

In some embodiments, the therapeutically active agent can be selected from at least one of IL-6 Inhibitors such as sarilumab, tocilizumab, RG6179, pharmaceutically acceptable salts thereof and combinations thereof, and/or HtRA1 Inhibitors such asIC-500, FHTR.2163, RG6147, pharmaceutically acceptable salts thereof and combinations thereof; and/or RASP Inhibitors such as reproxalap and pharmaceutically acceptable salts thereof; and/or rho kinase inhibitors such as netardusil, ripasudil, HA-1077, Y-27632, H-1152P, INS-I 15644, Y-39983, SB772077BS, LX7 1 Dl, AR-12286, AMA-0076, AR-13533, pharmaceutically acceptable salts thereof and combinations thereof; or plasma kallikrein inhibitors such as ecallantide, lanadelumab, berotralstat, ATN-249, KVD900, KVD824, THR-149, pharmaceutically acceptable salts thereof and combinations thereof, and/or nitric oxide donating PgAs such as Latanoprostene Bunod, NCX470, pharmaceutically acceptable salts thereof and combinations thereof; or mast cell stabilizers such aslodoxamide, nedocromil, pemirolast, cromolyn (e.g., chromolyn sodium), pharmaceutically acceptable salts thereof and combinations thereof, and/or IGF-1 R inhibitors such as teprotutumab, VRDN-001, VRDN-002, VRDN-003, ganitumab, figitumumab, MEDI-573, cixutumumab, dalotuzumab, robatumumab, AVE!642, BIIB022, xentuzumab, istiratumab, linsitinib, picropodopbyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, B11885578, B1893923, TT-100, XL-228, A-928605, pharmaceutically acceptable salts thereof and combinations thereof.

In some embodiments, the therapeutically active agent can be selected from at least one of TRPV1 antagonists such as asivatrep, V116517, fused azabicyclic, heterocyclic, and amide compounds as described, for example, in U.S. Patent Application No. 2004/0157849, U.S. Patent Application No. 2004/0209884, U.S. Patent Application No. 2005/0113576, International Patent Application No. WO 05/016890, U.S. Patent Application No. 2004/0254188, U.S. Patent Application No. 2005/0043351, International Patent Application o. WO 05/040121, U.S. Patent Application No. 2005/0085512, and Gomtsyan et al., 2005, J. Med. Chem. 48:744-752; fused pyridine derivatives as described, for example, in U.S. Patent Application No. 2004/0138454; pyridyl piperazinyl ureas as described, for example, in Swanson et al., 2005, J. Med. Chem. 48:1857-1872 and U.S. Patent Application No. 2005/0049241, as well as AMG8163 (Bannon et al., 2005, 11.sup.th World Congress on Pain) and BCTC (Sun et al., 2003, Chem. Lett. 13:3611-3616); 2-(piperazine-1-yl)-1H-Benzimidazole; pyridazinylpiperazines; urea derivatives as describe, for example, in U.S. Patent Application No. 2005/0107388, U.S. Patent Application No. 2005/018729 1, and U.S. Patent Application No. 2005/0154230, as well as A-425619 (El Kouhen et al., 2005, J. Pharmacol. Exp. Tuer. 314:400-409); cinnamides, including SB-366791 (Gunthorpe et al., 2004, Neuropharmacology 46:133-149) and AMG 9810 (Gawa et al., 2005, J. Pharmacol. Exp. Ther. 313:474-484). TRPV-1 antagonists may also include capsazepine, (E)-3-(4-t-butylphenyl)-N-(2,3-dihydrobenzo[b][1,4)dioxin-6-yl)acrylamide (commercially available for example as AMG981 O from Tocris Bioscience, Bristol, United Kingdom), and 4-tertiary butyl cyclohexane (commercially available as SYMSITIVE 1609 from Syrmise GmbH of Holzminden, Germany, as well as TRPVI antagonists as disclosed in U.S. Pat. Nos. 8,815,930, 6,933,311, 7,767,705 and U.S. Pat. App. Pub. Nos. 2010/0249203 and 2011/0104301, International Application WO/2008/013861; and/or AMG-517 and AMG-628 (Amgen Inc., Thousand Oaks, Calif.). TRPVI antagonists useful in the present invention are also described, for example, in International Patent Application No. WO 2006065484; International Patent Application No. WO 2003070247; U.S. Patent Application No. US 2005080095; and International Patent Application No. WO 2005007642. Additional TRPV1 antagonists useful in the methods and compositions and devices as disclosed herein include TRPV1 antagonists: ABT-102, AMG8562, AMG9810, BCTC, SB36679 1, JNJl 7203212, I-TIX, JYL-1421, A-425619, N-[4-[6-[4(Trifluoromethyl)phenyl)pyrimictin-4-yloxy]benzothiazol-2-yl]acetamide (also known as AL-49975 or AMG-517), (R)—N-(4-(6-(4-(1-(4-fluorophenyl)ethyl)piperazin-1-yl)pyrimidin-4-yloxy)benzo[d]thiazol-2-yl)acetamide (AL-49976, also known as AMG-628), pharmaceutically acceptable salts thereof and combinations thereof, e.g., 1-(2-(3,3-dimethylbutyl)-4-(trifluoromethyl)benzyl)-3-(1-methyl-1H-indazol-4-yl)urea; methyl 2,2-dimethyl-4-(2-((3-(1-methyl-1H-indazol-4-yl)ureido)methyl)-5-(trifluoromethyl)phenyl)butanoate; 1-(2-(4-hydroxy-3,3-dimethylbutyl)-4-(trifluoromethyl)benzyl)-3-(I-methyl-1H-indazol-4-yl)urea; 2,2-dimethyl-4-(2-((3-(1-methyl-1H-indazol-4-yl)ureido)methyl)-5-trifluor-methyl)phenyl)butanoic acid; 1-[4-Chloro-3-(3,3-dimethylbutyl)benzyl]-3-(1-methyl-1H-indazol-4-yl)urea-; 1-(2-isobutyl-4-(trifluoromethyl)benzyl)-3-(1-methyl-1H-indazol-4-yl)urea; 1-(2-isopropyl-4-(trifluoromethyl)benzyl)-3-(1-methyl-1H-indazol-4-yl)urea; 1-(4-Chloro-3-isopropylbenzyl)-3-(I-methyl-I H-indazol-4-yl)urea, pharmaceutically acceptable salts thereof and combinations thereof.

In some embodiments, the therapeutically active agent can be selected from at least one of TrkA antagonists including VM902A, Larotrectinib, Entrectinib, Selitrectinib (LOXO-195, BAY 2731954), repotrectinib (TPX-0005), pharmaceutically acceptable salts thereof and combinations thereof.

In some embodiments, the therapeutically active agent can be selected from at least one of lipophilic active agents such as betamethasone, bevacizumab (avastin), ciprofloxacin HCl, cortisone, cyclosporin, dexamethasone, ketoprofen, ketorolac, salicylic acid, sirolimus, sorafenib, sunitinib maleate, tacrolimus; and/or betaxolol, indomethacin, propranolol, fluconazole, fluorometholone, timolol, ethoxzolamide, hydrocortisone cabozantinib, Axitinib, tivozanib.

In certain embodiments, the therapeutically active agent may be a combination of drugs, for example for combination therapy purposes. Combinations of active agents may be co-administered in a drug delivery system, for example an implant, or may be included as a bispecific molecule. Exemplary combinations useful in the drug-delivery systems of the present invention include complement inhibitors in combination with anti-VEGF agents, which may be used, for example, in treating dry AMD/GA and wet AMD in patients that have both, and to prevent one of those diseases from occurring. Such a combination may be used to treat patients with wet AMD without GA, to prevent or delay developing GA after they received a combination of anti-VEGF and a complement agent. Example of combinations of complement inhibitors and anti-VEGF agents include Aflibercept+Pegcetacoplan, Aflibercept+Avacincaptad Pegol, Ranibizumab+Pegcetacoplan, Ranibizumab+Pegcetacoplan, Axitinib+pegcetacoplan, Axitinib+Avacincaptad pegol, Vorolanib+Pegcetacoplan, Vorolanib+Avacincaptad pegol, Lenvatinib+Pegcetacoplan, Lenvatinib+Avacincaptad, Faricimab+Pegcetacoplan, Faricimab+Avacincaptad Pegol, Bevacizumab+Pegcetacoplan, Bevacizumab+Avacincaptad Pegol.

In other embodiments, the combination of active agents may include Anti-VEGF and IL-6 blockers such as any combination of Aflibercept, Ranibizumab, Bevacizumab, Faricimab, Axitinib, Vorolanib, Lenvatinib (Anti-VEGF) with Sarilumab, Tocilizumab, RG6179 (IL-6 blocker).

In other embodiments, the combination of active agents may include Beta Blocker in combination with a PgA analog such as Timolol (most commonly used in glaucoma) and a PgA analog such as any one of Latanoprost, bimatoprost, travoprost.

Active agent combinations in the present invention may also include combinations of at least one therapeutically active agent with at least one diagnostically active agent, or combinations of more than two active agents.

Diagnostically active agents may be, e.g., imaging agents, markers, or visualization agents. Generally, diagnostic agents may be substances used to examine the body in order to detect impairment of its normal functions. In some cases, diagnostic agents may be agents with a functional purpose, such as for use in the detection of ocular deformities, ailments, and pathophysiological aspects. For example, the diagnostic agent may be an important and effective diagnostic adjuvant, such as a dye (e.g., fluorescein dye, indocyanine green, trypan blue, a dark quencher such as a cyanine dye, an azo dye, an acridine, a fluorene, an oxazine, a phenanthridine, a naphthalimide, a rhodamine, a benzopyrone, a perylene, a benzanthrone, pra benzoxanthrone), to aid in visualization of ocular tissues. The diagnostic agent may comprise paramagnetic molecules, fluorescent compounds, magnetic molecules, radionuclides, x-ray imaging agents, and/or contrast media. In some embodiments, a diagnostic agent may include radiopharmaceuticals, contrast agents for use in imaging techniques, allergen extracts, activated charcoal, different testing strips (e.g., cholesterol, ethanol, and glucose), pregnancy test, breath test with urea 13C, and various stains/markers. In some embodiments, the labelling moiety is a fluorescent dye or a dark quencher, selected from the group consisting of a coumarin, a cyanine dye, an azo dye, an acridine, a fluorene, an oxazine, a phenanthridine, a naphthalimide, a rhodamine, a benzopyrone, a perylene, a benzanthrone, and a benzoxanthrone. In particular non-limiting embodiments, the fluorescent dye is or is the residue of a compound selected from the group consisting of Coumarin, Fluorescein, Cyanine 3 (Cy3), Cyanine 5 (Cy5), Cyanine 7 (Cy7), Alexa dyes, bodipy derivatives, (E)-2-(4-(phenyldiazenyl)phenoxy)acetic acid, 3-(3′,3′-dimethyl-6-nitrospiro[chromene-2,2′-indolin]-1′-yl)propanoate (Spiropyran), 3,5-dihydroxybenzoate and (E)-2-(4-(phenyldiazenyl)phenoxy)acetic acid, or combinations thereof.

In embodiments of the invention, the active agent is a drug in the form of a liquid oil at temperatures up to 37° C., e.g., travoprost, etc., which forms at least part of the hydrophobic organic liquid or may even be used instead of the hydrophobic organic liquid.

According to certain embodiments of the invention the active agent may be oil soluble and dissolved in the hydrophobic organic liquid, or the active agent is oil insoluble and can be dispersed in particle form in the hydrophobic organic liquid, or emulgated in liquid form.

In embodiments where the active agent is used in particulate form, the active agent particles may be micronized particles, e.g., having a D50 particle size of less than about 15 μm, or less than 10 m and/or a D99 particle size of less than about 100 μm, or less than about 50 m, or a D90 particle size of about 50 μm or less, or 5 μm or less and/or a D98 particle size of about 10 μm or less. In other embodiments the active agent particles may be nanosized particles, e.g., having a D50 particle size of less than about 100 nm, or less than about 50 nm, and/or a D99 particle size of less than about 50 nm, or a D90 particle size of about 5 nm or less and/or a D98 particle size of about 10 nm or less. Particle sizes are determined as disclosed in the “Definitions” section herein.

### Composition Ranges

According to the present invention, the organogel of the drug-delivery system may be designed as desired for the intended use and therapeutic application. Typically the organogel comprises from 1 to 90 wt.-% of the hydrophobic organic liquid, or 5-90 wt.-%, 5-60 wt.-%, 10-50 wt.-%, 10-40 wt.-%, 15-40 wt.-%, or 15-35 wt. %; from 5 to 95 wt.-% of the covalently crosslinked polymer network, or 10-95 wt.-%, 40-95 wt.-%, 50-90 wt.-%, 60-90 wt.-%, or 60-85 wt.-%; and from 1 to 50 wt.-% of the active agent, or 5-50 wt.-%, 5-40 wt.-%, 10-30 wt.-%, or 10-25 wt.-%; wherein all weight percentages are selected to amount to 100% in total, and the wt.-% is based on the total dry weight of the organogel or drug-delivery system, respectively.

### Method of Manufacture

According to the invention, a method of manufacturing the sustained release, biodegradable drug-delivery system as described herein is provided. In certain embodiments, the method for manufacturing a sustained release, biodegradable drug-delivery system comprises forming an organogel from at least a covalently crosslinked polymeric network, a hydrophobic organic liquid, optionally a solvent, and at least one active agent, wherein the hydrophobic organic liquid and the active agent are contained, e.g., immobilized, in the biodegradable, covalently crosslinked polymeric network, shaping the organogel, and optionally removing the solvent from the organogel.

In a particular procedure, the step of forming the organogel (step (1)) comprises providing (a) the hydrophobic organic liquid; (b) the at least one active agent; (c) a first covalently crosslinkable precursor comprising first functional groups; (d) a second covalently crosslinkable precursor comprising second functional groups; combining all these in any suitable sequence into a reaction mixture; and (e) allowing the reaction mixture to gel by forming a covalently crosslinked polymer network.

Optionally at least one organic solvent may be added to any of (a), (b), (c), (d) and (e), and removed after the organogel has formed.

In one embodiment, in (c) at least one first multi-arm precursor is provided. Precursors and multi-arm precursors used in the present invention have been described in detail in the section under the heading “Precursor components”. In some embodiments, the at least one multi-arm precursor comprises at least 8 arms, or at least 4 arms. The at least one multi-arm precursor comprises an electrophile or a nucleophile as the first functional group.

In another embodiment, the at least one first multi-arm precursor comprises at least two multi-arm precursors. In such embodiments, one multi-arm precursor comprises an electrophile and another multi-arm precursor comprises a nucleophile as the first or second functional groups. In another embodiment, the at least one multi-arm precursor comprises at least one multi-arm precursor comprising an electrophile or a nucleophile as the first functional group and a small molecule crosslinker comprising an electrophile or a nucleophile as the second functional group.

In these embodiments, the nucleophile can be an amine such as a primary amine, a thiol, a dibenzocyclooctyne, or a hydrazide, and the electrophile can be succinimidyl esters, succinimidyl carbonates, nitrophenyl carbonates, aldehydes, ketones, acrylates, acrylamides, maleimides, vinylsulfones, iodoacetamides, alkenes, alkynes, azides, norbornenes, epoxides, mesylates, tosylates, tresyls, cyanurates, orthopyridyl disulfides, or halides. In an embodiment of the invention, if the electrophile is a succinimidyl ester, it may comprise a reactive group such as succinimidyl succinate (SS), succinimidyl glutarate (SG), succinimidyl adipate (SAP), succinimidyl azelate (SAZ), or succinimidyl glutaramide.

In some embodiments, the at least one multi-arm precursor is a first multi-arm PEG precursor comprising a primary amine, or a first multi-arm PLGA precursor comprising a primary amine. The first covalently crosslinkable precursor comprising first functional groups may thus be hydrophilic (PEG) or hydrophobic (PLGA).

In one embodiment, in (d) at least one further, second multi-arm precursor is provided. Precursors and multi-arm precursors used in the present invention have been described in detail in the section under the heading “precursor components”. In some embodiments, the at least one second multi-arm precursor comprises at least 8 arms, or at least 4 arms. The at least one second multi-arm precursor may comprise an electrophile or a nucleophile as the second functional group.

In another embodiment, the at least one second multi-arm precursor comprises at least two multi-arm precursors. In such embodiments, one multi-arm precursor comprises an electrophile and another multi-arm precursor comprises a nucleophile. In another embodiment, the at least one second multi-arm precursor comprises at least two multi-arm precursors each comprising an electrophile.

In all these embodiments, the nucleophile can be an amine such as a primary amine, a thiol, a dibenzocyclooctyne, or a hydrazide, and the electrophiles can be succinimidyl esters, succinimidyl carbonates, nitrophenyl carbonates, aldehydes, ketones, acrylates, acrylamides, maleimides, vinylsulfones, iodoacetamides, alkenes, alkynes, azides, norbornenes, epoxides, mesylates, tosylates, tresyls, cyanurates, orthopyridyl disulfides, or halides. Besides classical electrophile-nucleophile condensation reactions other chemical reaction types based on electrophiles and nucleophiles may also be used in the present invention. For example, with azide and dibenzocyclooctyne functionalization, precursors may be crosslinked via so-called click-chemistry reactions (cf. H. C. Kolb; M. G. Finn; K. B. Sharpless (2001). “Click Chemistry: Diverse Chemical Function from a Few Good Reactions”, Angewandte Chemie International Edition, 40 (11): 2004-2021).

In some embodiments, the at least one second multi-arm precursor comprises at least two second multi-arm precursors comprising a first multi-arm precursor comprising an electrophile comprising a first reactive group and a second multi-arm precursor comprising an electrophile comprising a second reactive group. In an embodiment of the invention, if the electrophile is a succinimidyl ester, the first and the second reactive groups are selected from succinimidyl succinate (SS), succinimidyl glutarate (SG), succinimidyl adipate (SAP), succinimidyl azelate (SAZ), or succinimidyl glutaramide.

At least one of the first or second crosslinkable precursors has a functionality of greater than 2, such as 3 to 10, or 3 to 9, or 4 to 8, or 4, and the first crosslinkable precursor may be a dendrimer or a multi-arm precursor having a core and from 2 to 12 arms, or 3 to 10 arms, 4 to 8 arms, or 4 or 8 arms, each arm comprising a polymeric unit as defined herein and having a terminus bearing the first or second functional group. For example, a 4 arm precursor may be derived from pentaerythritol or ethylenediamine, comprising 4 arms of polymer units attached to it. In some embodiments the arms comprise hydrophobic polymer units selected from polylactic acid (PLA) units and polylactic-co-glycolic acid (PLGA) units, or combinations thereof. In some embodiments the arms comprise hydrophilic polymer units selected from polyethylene glycol (PEG), polypropylene glycol (PPG), and polyglycolic acid (PGA), or combinations thereof. The second crosslinkable precursor may be a non-polymer crosslinker, preferably a small molecule amine such as tris(2-aminoethyl)amine (TAEA) or trilysine.

When PLGA units are used, the ratio of polylactic-co-glycolic acid (PLGA) precursors to polyethylene glycol (PEG) precursors may be set to about 2.5:1 to about 1:2.5, or about 2:1 to 1:2, or about 1:1. Furthermore, the polylactic-co-glycolic acid (PLGA) precursors may have an L/G ratio (in % L or G blocks) ranging from about 1:99 to about 99:1, or about 10:90 to about 90:10, or about 25:75 to about 75:25, or about 50:50. The L/G ratio of the polylactic-co-glycolic acid (PLGA) units can be selected to adjust the hydrophobicity of the polymeric network and to provide a sustained release of the active agent from the organogel. Additionally, or alternatively, the ratio of the amounts of the first to second crosslinkable precursors may be selected to adjust the hydrophobicity of the polymeric network and to provide a sustained release of the active agent.

In certain embodiments, each of (a), (b), (c) and (d) above are then processed to obtain (e) and (f). In an embodiment, before (e) either the first precursor may be premixed with the hydrophobic organic liquid, or the second precursor may be premixed with the hydrophobic organic liquid, and one or more solvents may optionally be added to any of these premixtures, and the active agent of (b) may be added to any of these premixtures or added to the reaction mixture in (e).

In one embodiment when all components have been combined in the reaction mixture (e), at least two precursors react in an electrophile-nucleophile reaction to form a covalently cross-linked matrix that is an organogel. The reaction may be initiated or promoted by heating, or can occur at ambient conditions.

The step of shaping the organogel (step (2)) may comprise molding or extruding or casting the reaction mixture prior to complete gelling of the organogel, then allowing the mixture to gel, and optionally removing the solvent. Molding may be done by filling the reaction mixture into a mold or tubing prior to complete gelling of the organogel, allowing the mixture to gel, and optionally removing the solvent. In some embodiments the reaction mixture may be filled into a fine diameter tubing or needle in order to prepare an organogel strand. The reaction mixture may also be applied as a coating on a substrate. As part of the process, the cured organogel can be deformed and rigidified to allow injection through a needle lumen, the rigidity and reshaping being reversible upon contacting the warmth and/or moisture of tissue. Rigidity could be provided through crystallization, a secondary crosslink mechanism, or a water soluble temporary structural component, e.g., PEG fibers.

A composition (e) with the precursors mixed therein can be made with viscosity suitable for introduction through a small gauge needle using manual force. A small gauge needle has a diameter less than the diameter of a needle with a gauge of 27, e.g., 28, 29, 30, 31, 32, or 33 gauge, with the gauge being specific for inner and/or outer diameters. Moreover, hollow-tube wires, as used in the intravascular arts, may be used to deliver the materials to an implantation site for forming the drug-delivery device in situ, including those with inner and/or outer diameters equivalent to the small gauge needles, or smaller. Thus, a viscosity of between about 1 to about 100,000 mPa-s may be used; artisans will immediately appreciate that all the ranges and values within the explicitly stated ranges are contemplated e.g., about 10 to about 10,000 mPa-s, less than about 5 to about 10,000 mPa-s, less than about 100 or about 500 mPa-s, or between about 1 and about 100 mPa-s. The viscosity may be controlled, e.g., by choosing appropriate precursors, adjusting solids and or solvent concentrations, and reaction kinetics. In general, lower concentrations of precursors, increased hydrophilicity, lower molecular weights favor a lower viscosity.

Viscosity enhancers may be used in conjunction with precursors. In certain embodiments, the viscosity enhancers do not react with the precursors to form covalent bonds. While it is appreciated that precursors that are generally free of such bonding may sometimes participate in unwanted side reactions, these have little effect on the organogel so that the precursors are “free” of such reactions. For instance, if the precursors react by electrophile-nucleophile reactions, the viscosity enhancers may be free of electrophiles or nucleophiles that can form covalent bonds with functional groups of the precursors, even if there is some low level of unwanted side reactions. Viscosity enhancers may be hydrophilic polymers with a molecular weight, e.g., of at least 20,000, or from about 10,000 to about 500,000 Daltons; artisans will immediately appreciate that all values and ranges between these explicitly stated values are described, e.g., at least about 100,000 or 200,000. A concentration of about 1% to about 40%, or about 5% to about 25% w/w may be used, for instance. PEG (e.g., M.W. 100,000 to 250,000) is useful, for example. Viscosity enhancers may be free of electrophiles and/or nucleophiles. Viscosity enhancers may be free of one or more functional groups such as hydroxyl, carboxyl, amine, or thiol. Viscosity enhancers may include one or more biodegradable links as described herein for precursors. Viscosity enhancers can be useful to prevent precursors from running-off a tissue site before the precursors crosslink to form a gel.

### Sustained Release Kinetics:

In certain embodiments, the use of an organogel in the sustained release, biodegradable drug-delivery system of the invention allows to modify the release of an active agent from the drug-delivery system by several measures. For example, tailoring or suitably selecting the precursor components forming the crosslinked polymer network according to their hydrophilic and/or hydrophobic properties may have an influence on active agent release. Furthermore, the release of an active agent from the drug-delivery system may be modified or controlled by suitably selecting the hydrophobic organic liquid according to its properties such as one or more of hydrophobicity, viscosity, compatibility with the active agent, solubility or insolubility of the active agent in the hydrophobic organic phase, and the like.

Accordingly, in various embodiments of the present invention the selection of the hydrophobic liquid, and/or the hydrophobicity of the polymeric network, and/or the L/G ratio may be used to tune the release rate. Each of these individual parameters can be selected alone or in combination with each other to provide for the controlled release of the active agent.

In certain embodiments, the sustained release drug-delivery system of the present invention is formulated to make an active agent available over an extended period of time, thereby allowing a reduction in dosing frequency compared to an immediate release dosage form, such as for example a solution of an active agent that is topically applied onto the eye (i.e., eye drops). In certain embodiments, the release of the active agent comprises constant active agent release, tapered active agent release as well as any combination thereof such as a constant active agent release followed by a tapered active agent release. The “sustained release” may be measured in vitro in an aqueous solution under physiological conditions such as at pH 7.2-7.4 and 37° C. and is considered to be the same or substantially the same when the drug-delivery system is administered in vivo to a subject.

In various embodiments of the present invention the active agent release follows zero order release kinetics or substantially zero order release kinetics, preferably without a “burst” of active agent at the beginning of the period.

Embodiments of the present invention may provide for a release of a therapeutically effective amount of the active agent for a period of time, such as up to 1 year, up to 9 months, up to 6 months, up to 3 months, up to 1 month, or up to about 25 days after administration. Other embodiments of the present invention may provide for a release of a therapeutically effective amount of the active agent of up to about 14 days, or up to about 21 days after administration, or a release of a therapeutically effective amount of the active agent for a period of about 6 hours or longer after administration, or for a period of about 12 hours, or 24 hours or longer or about 48 or longer, or about 72 hours or longer or about 7 days or longer, or about 10 days or longer after administration. The present invention contemplates all of the above lower and higher time periods in any combination of ranges.

In various embodiments of the present invention the organogel delays the release of a water-soluble active agent or accelerates the release of a hydrophobic active agent.

In one aspect of the present invention, a sustained release drug-delivery system such as a pharmaceutically acceptable implant is provided for a controlled release of the active agent comprised therein (e.g., the total amount). Throughout this section, controlled release is to be considered as the controlled release measured from the time and under conditions wherein the implant is first immersed in an aqueous solution under physiological conditions such as at pH 7.2-7.4 and temperature 37° C. After exposure to physiological conditions, the organogel comprised in the drug-delivery system may slowly release the hydrophobic organic liquid from the organogel and concomitantly forms a hydrogel.

According to certain aspects of the invention, a sustained release drug-delivery system such as a pharmaceutically acceptable implant is provided for a controlled release of the total amount of the active agent comprised therein. In certain embodiments, the controlled release can be characterized as the amount of the active agent released on day 1 is from 0 to 50% of the total amount of the active agent, the amount of the active agent released per day from day 2 until the last day of release is from 0 to 50% of the total amount of the active agent, and/or the number of days required for 100% release of the total amount of the active agent is at least 2 days.

In certain embodiments, the controlled release can be characterized as the amount of the active agent released on day 1 is from 0 to 50% of the total amount of the active agent, the amount of the active agent released per day from day 2 until the last day of release is from 0 to 50% of the total amount of the active agent, and/or the number of days required for 100% release of the total amount of the active agent is at least 3 days.

In certain embodiments, the controlled release can be characterized as the amount of the active agent released on day 1 is from 0 to 50% of the total amount of the active agent, the amount of the active agent released per day from day 2 until the last day of release is from 0 to 50% of the total amount of the active agent, and/or the number of days required for 100% release of the total amount of the active agent is at least 4-7 days.

In certain embodiments, the controlled release can be characterized as the amount of the active agent released on day 1 is from 0 to 50% of the total amount of the active agent, the amount of the active agent released per day from day 2 until the last day of release is from 0 to 50% of the total amount of the active agent, and/or, the number of days required for 100% release of the total amount of the active agent is at least 10-15 days.

In certain embodiments, the controlled release can be characterized as the amount of the active agent released on day 1 is from 0 to 50% of the total amount of the active agent, the amount of the active agent released per day from day 2 until the last day of release is from 0 to 50% of the total amount of the active agent, and/or the number of days required for 100% release of the total amount of the active agent is at least 10-30 days.

In certain embodiments, the controlled release can be characterized as the amount of the active agent released on day 1 is from 0 to 50% of the total amount of the active agent, the amount of the active agent released per day from day 2 until the last day of release is from 0 to 50% of the total amount of the active agent, and/or the number of days required for 100% release of the total amount of the active agent is greater than 30 days.

According to certain embodiments of the invention, the controlled release is characterized by: the amount of the active agent released on day 1 is from 0 to 25%, 0 to 20% 0 to 10%, 0 to 5%, or about 0% of the total amount of the active agent, the amount of the active agent released per day from day 2 until the last day of release is from 0 to 50% or from 0 to 40% or from 0 to 30% or from 0 to 20% or from 0 to 10% or from 0 to 5% of the total amount of the active agent. In certain embodiments, the number of days required for 100% release of the total amount of the active agent is at least 3 days but no greater than 30 days, 25 days, or no greater than 16 days. In other embodiments the time is as disclosed above.

In one embodiment, the controlled release characterized above comprises a zero-order release, such as near zero order release, or substantially zero order release. In one embodiment, the zero-order release or near zero order release or substantially zero order release begins at least 1 day after the pharmaceutically acceptable implant has been immersed under physiological conditions such as pH 7.2-7.4 and 37° C.

A dosage form or implant exhibiting zero order release rate would exhibit a relatively straight line in a graphical representation of percent active agent released versus time. In certain embodiments of the present invention, the zero-order release is accomplished over the entire period of release. In certain embodiments of the present invention, the zero-order release is accomplished over a part of the period of release. In certain such embodiments the zero-order release is accomplished from the end of day 1, i.e., from 24 hours after the start of the release, to the end of the release. If less or no release is accomplished before the end of day 1 such release would be considered to have a lag time for one day or 24 hours. Such a lag time could also be longer. If a high release is accomplished before the end of day 1 such release would be considered to have a burst during the first day or 24 hours. Such a burst time could also be longer. Zero order release can also be accomplished during the entire period of release. The entire period of release is, in this context, defined until 95% of the release is accomplished.

Zero order release is defined to be accomplished, within the meaning of the present invention, if during the respective time the release is proportional to elapsed time. Proportional to elapsed time means that the proportional release is calculated based on the entire time of the zero order release defining a straight line (release in % cumulative release during the entire period of time during which zero order is accomplished divided by said entire period of time defining a straight line) and the release at any time point in between, i.e., start of zero order release and end of zero order release is within 20% points of the % cumulative release of said proportional release defined by said straight line.

### Administration

The drug-delivery system of the present invention may be in the form of an implant, such as a medical implant or a pharmaceutically acceptable implant, an implant coating, or an oral dosage form, etc. The drug-delivery system may also be provided in the form of a kit as further defined herein below, for example for forming an implant in situ.

If the sustained release, biodegradable drug-delivery system is an implant, the implant may be one of an intraocular implant, intracaveal implant, intracameral implant, an implant for introduction into the anterior chamber, the vitreous, episcleral, in the posterior subtenon's space (Inferior fornix), subconjunctival, intracameral, peribulbar, retrobulbar, sub-tenon, retinal, subretinal, intracanalicular, intravitreal, intrasceleral, choroidal, suprachoroidal, a retina, subretinal, or a lens, a surface of the cornea or the conjunctiva, puncta (canaliculus, upper/lower canaliculus), ocular fornix, upper/lower ocular fornix, subtenon space, choroid, suprachoroid, tenon, cornea, cancer tissue, organ, prostate, breast, joint space, subdural, dental, subcutaneous, carpal tunnel, perivascular, surgically created space or injury, void space, and potential space.

In embodiments of certain embodiments of the invention, the sustained release, biodegradable drug-delivery system may be formulated for administration via diverse routes such as oral, parenteral, or by operative insertion or injection. Oral dosage forms may consist of the organogel of the present invention, which may optionally be enterically coated, or in the form of small particulate forms filled into capsules and the like.

### Treatment Methods

According to the invention, the sustained release, biodegradable drug-delivery system is configured for use as a medicament, such as for use in treating a disease or medical condition of a patient, the method comprising forming an organogel comprising a therapeutically active agent dispersed in a hydrophobic organic liquid that is contained in a covalently crosslinked polymeric network, wherein the organogel is formed in situ at a treatment site of the patient, or is prefabricated and delivered to or implanted at a treatment site of the patient in order to release the active agent over an extended period of time.

Provided are methods for treating a disease or medical condition of a patient comprises forming an organogel comprising a therapeutically active agent dispersed in a hydrophobic organic liquid that is contained in a covalently crosslinked polymeric network, wherein the organogel is formed in situ at a treatment site of the patient, or is prefabricated and delivered to or implanted at a treatment site in order to release the active agent over an extended period of time. The method for treating a disease or medical condition of a patient may comprise administering an organogel comprising a therapeutically active agent dispersed in a hydrophobic organic liquid that is contained in a covalently crosslinked polymeric network to the patient in order to release the active agent over an extended period of time.

The treatment site may be one of the anterior chamber, the vitreous, episcleral, in the posterior subtenon's space (Inferior fornix), subconjunctival, intracameral, peribulbar, retrobulbar, sub-tenon, retinal, subretinal, intracanalicular, intravitreal, intrasceleral, choroidal, suprachoroidal, a retina, subretinal, or a lens, a surface of the cornea or the conjunctiva, puncta (canaliculus, upper/lower canaliculus), ocular fornix, upper/lower ocular fornix, subtenon space, choroid, suprachoroid, tenon, cornea, cancer tissue, organ, prostate, breast, joint space, subdural, dental, subcutaneous, carpal tunnel, perivascular, surgically created space or injury, void space, and potential space.

In embodiments of the invention, the disease or medical condition to be treated is an eye disease, particularly back-of-the-eye diseases such as any ocular disease of the posterior segment that affects the vasculature and integrity of the retina, macula or choroid leading to visual acuity disturbances, loss of sight or blindness, particularly disease states of the posterior segment resulting from age, trauma, surgical interventions, such as age-related macular degeneration (AMD) cystoid macular edema (CME), diabetic macular edema (DME), posterior uveitis, and diabetic retinopathy; or glaucoma, ocular hypertension, hyphema, presbyopia, cataract, retinal vein occlusion, inflammation. The ocular disease may be selected from retinal neovascularisation, choroidal neovascularisation, Wet AMD, Dry AMD, retinal vein occlusion, diabetic macular edema, retinal degeneration, corneal graft rejection, retinoblastoma, melanoma, glaucoma, autoimmune uveitis, uveitis, proliferative vitreoretinopathy, and corneal degeneration, acute and chronic macular neuroretinopathy, central serous chorioretinopathy, macular edema, acute multifocal placoid pigment epitheliopathy, Behcet's disease, birdshot retinochoroidopathy, posterior uveitis, posterior scleritis, serpiginous choroiditis, subretinal fibrosis, uveitis syndrome, Vogt-Koyanagi-Harada syndrome, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, carotid artery disease (CAD), frosted branch angiitis, sickle cell retinopathy, angioid streaks, familial exudative vitreoretinopathy, Eales disease, proliferative vitreal retinopathy, diabetic retinopathy, retinal disease associated with tumors, congenital hypertrophy of the retinal pigment epithelium (RPE), posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors, myopic retinal degeneration, acute retinal pigment epithelitis, glaucoma, endophthalmitis, cytomegalovirus retinitis, retinal cancers, retinitis pigmentosa, Leber's Congenital Amaurosis, Choroideremia, X-linked retinitis pigmentosa, best vitelliform macular dystrophy, x-linked retinoschisis, achromatopsia CNGA3, achromotopsia CNGB3, LHON, Stargardt disease, Usher syndrome, Norrie disease, Bardet-Biedl syndrome, and red-green color blindness.

In the treatment methods of the present invention the sustained release, biodegradable drug-delivery system as discussed in a separate section herein is administered to a subject or patient. This drug-delivery system is for controlled release of any active agent that has been discussed in a separate section herein. The controlled release is also defined herein in a separate section.

The methods described in this section can also comprise administration of the drug-delivery system such as a pharmaceutically acceptable implant in combination with another agent also termed combination therapy.

In one embodiment, the combination therapy comprises administering a pharmaceutically acceptable implant of the invention in combination with one or more additional agents either on the same or different day. In one embodiment, the additional agent to be administered in a combination therapy can be a liquid formulation of the agent, or it may be comprised in an oral dosage form. Thus, the additional agent can be any small molecule, large molecule, a protein, a nanoparticle, or any other of the active agents described herein.

In certain combination therapy embodiments, the therapeutically active agent may be a combination of drugs. Combinations of active agents may be co-administered by including all active agents in a drug delivery system, for example an implant, or may be included as a bispecific molecule. Exemplary combinations useful in the drug-delivery systems of the present invention include complement inhibitors in combination with anti-VEGF agents, which may be used, for example, in treating dry AMD/GA and wet AMD in patients that have both, and to prevent one of those diseases from occurring. Such a combination may be used to treat patients with wet AMD without GA, to prevent or delay developing GA after they received a combination of anti-VEGF and a complement agent. Examples of combinations of complement inhibitors and anti-VEGF agents include Aflibercept+Pegcetacoplan, Aflibercept+Avacincaptad Pegol, Ranibizumab+Pegcetacoplan, Ranibizumab+Pegcetacoplan, Axitinib+pegcetacoplan, Axitinib+Avacincaptad pegol, Vorolanib+Pegcetacoplan, Vorolanib+Avacincaptad pegol, Lenvatinib+Pegcetacoplan, Lenvatinib+Avacincaptad, Faricimab+Pegcetacoplan, Faricimab+Avacincaptad Pegol, Bevacizumab+Pegcetacoplan, Bevacizumab+Avacincaptad Pegol.

In other embodiments, the combination of active agents may include Anti-VEGF and IL-6 blockers such as any combination of Aflibercept, Ranibizumab, Bevacizumab, Faricimab, Axitinib, Vorolanib, Lenvatinib (Anti-VEGF) with Sarilumab, Tocilizumab, RG6179 (IL-6 blocker).

In other embodiments, the combination of active agents may include Beta Blocker in combination with a PgA analog such as Timolol (most commonly used in glaucoma) and a PgA analog such as any one of Latanoprost, bimatoprost, travoprost.

Active agent combinations in the present invention may also include combinations of at least one therapeutically active agent with at least one diagnostically active agent, or combinations of more than two active agents.

The method of treatment comprising administering the drug-delivery system such as a pharmaceutically acceptable implant as described in this section may comprise any one of intravitreal, intracameral, subconjunctival, retrobulbar, sub-tenon, subretinal, and suprachoroidal injections. The method of administration may also be topical or oral.

The active agent or the additional agent to be administered in a combination therapy, may also be a diagnostic agent. Diagnostic agents may be substances used to examine the body in order to detect impairment of its normal functions. In some cases, diagnostic agents may be agents with a functional purpose, such as for use in the detection of ocular deformities, ailments, and pathophysiological aspects. For example, the diagnostic agent may be an important and effective diagnostic adjuvant, such as a dye (e.g., fluorescein dye, indocyanine green, trypan blue, a dark quencher such as a cyanine dye, an azo dye, an acridine, a fluorene, an oxazine, a phenanthridine, a naphthalimide, a rhodamine, a benzopyrone, a perylene, a benzanthrone, pra benzoxanthrone), to aid in visualization of ocular tissues. The diagnostic agent may comprise paramagnetic molecules, fluorescent compounds, magnetic molecules, radionuclides, x-ray imaging agents, and/or contrast media. In some embodiments, a diagnostic agent may include radiopharmaceuticals, contrast agents for use in imaging techniques, allergen extracts, activated charcoal, different testing strips (e.g., cholesterol, ethanol, and glucose), pregnancy test, breath test with urea 13C, and various stains/markers. In some embodiments, the labelling moiety is a fluorescent dye or a dark quencher, selected from the group consisting of a coumarin, a cyanine dye, an azo dye, an acridine, a fluorene, an oxazine, a phenanthridine, a naphthalimide, a rhodamine, a benzopyrone, a perylene, a benzanthrone, and a benzoxanthrone. In particular non-limiting embodiments, the fluorescent dye is or is the residue of a compound selected from the group consisting of Coumarin, Fluorescein, Cyanine 3 (Cy3), Cyanine 5 (Cy5), Cyanine 7 (Cy7), Alexa dyes, bodipy derivatives, (E)-2-(4-(phenyldiazenyl)phenoxy)acetic acid, 3-(3′,3′-dimethyl-6-nitrospiro[chromene-2,2′-indolin]-1′-yl)propanoate (Spiropyran), 3,5-dihydroxybenzoate and (E)-2-(4-(phenyldiazenyl)phenoxy)acetic acid, or combinations thereof.

### Release Controlling Method

In one aspect the invention relates to method for controlling the release of an active agent from a sustained release, biodegradable drug-delivery system as described herein before, by selecting a combination of a hydrophobic organic liquid and an active agent dispersed therein, wherein either one or a combination of the following criteria applies:
a) the active agent dispersed in the hydrophobic liquid (e.g., oil) is released from the organogel together with the hydrophobic liquid (diffusion/absorbance rate of oil determines agent release rate);
b) the active agent is eluted from the hydrophobic liquid (e.g., oil) into the body directly, with the agent release rate being controlled by at least one of drug solubility and/or diffusivity in the hydrophobic liquid (e.g., oil) and/or surface area on the implant (agent release rate is largely independent of diffusion/absorbance rate of hydrophobic liquid).

In certain embodiments of the present invention, the release of the active agent is mainly controlled by diffusion of the active agent and/or the hydrophobic liquid (e.g., oil). The degradation rate of the polymer network offers another, independent additional mechanism for release control. In certain embodiments, the hydrophobic liquid delays or accelerates the degradation, which can be used as another method of control the release of the active agent. When the active agent dispersed in the hydrophobic liquid is released from the organogel together with the hydrophobic liquid, the release rate of the agent will be essentially influenced or determined by the diffusion rate of the oil into the surrounding tissue or bodily environment. In other embodiments, the active agent may diffuse out of the hydrophobic liquid more readily than the oil out of the polymer network.

When in contact with aqueous body fluids, the organogels of the present invention swell by taking up water. The degree of swelling is largely determined by the gel-forming components used and their hydrophobicity/hydrophilicity. Swelling may result in an increase of length and/or diameter dimensions of the organogels according to the invention of up to 2000%, 1000%, 100%, 95%, 90%, 80%, 75%, 70%, 60%, 50%, 40%, 30%, 20% or 10%.

However, even after swelling, in certain embodiments, the drug-delivery system of the present invention retains its shape or substantial shape over extended periods of time due to crosslinking of the polymer components. In certain embodiments, the polymer network of the organogel will only substantially degrade after all active agent has been released, or at least after most of the active agent, for example at least 50 wt.-%, 60 wt.-%, 70 wt.-%, 80 wt.-%, 90 wt.-% or 99 wt.-%, or 100 wt.-% of the active agent has been released.

In certain embodiments, it is believed that while swelling in the first place leads to an increase of the size of, e.g., an organogel implant, it also has an effect on active agent release and/or the biodegradation of the gel matrix. By swelling of the organogel, with increasing amounts of water intruding into the organogel, hydrophobic organic liquid may be forced out of the gel matrix, or out-diffusion thereof is accelerated, either together with the active agent dissolved therein, or without the active agent if it is undissolved in the hydrophobic liquid phase. and the hydrophobic liquid is subsequently replaced by water,

For example, water replacing the organic hydrophobic liquid in the gel over time may steadily dissolve hydrophilic active agents dispersed but not dissolved in the organic hydrophobic liquid, which can be used to control the active agent release. In this embodiment, the active agent release is mainly or completely controlled diffusion of the active agent through the oil and polymer into the surrounding tissue. In certain embodiments, another factor influencing or determining the release of the active agent dispersed in the hydrophobic liquid when the active agent release rate is largely independent of the diffusion rate of the hydrophobic liquid, for example when it is an hydrophilic agent dispersed as solid particles in the hydrophobic liquid, is the rate of diffusion of water into the gel and/or hydrophobic liquid, thereby subsequently dissolving and eluting the active agent from the organogel into the surrounding aqueous environment.

Furthermore, in certain embodiments, water slowly replacing the hydrophobic organic liquid slowly transforms the organogel into a hydrogel, which is still crosslinked so it maintains its shape, but may then be easier (bio-)degraded by hydrolysis and/or enzymatic reactions after the drug-delivery system is depleted of active agent and/or hydrophobic liquid.

The overall release of the active agent is controlled by at least one, or by a combination of all these release mechanisms.

Additionally, in certain embodiments, while maintaining or substantially maintaining its structure due to chemical cross-linking of individual polymer chains, the organogel during swelling by taking up becomes softer and more flexible to mimic natural tissue.

In a further aspect the invention relates to a method for controlling the release of an active agent from a sustained release, biodegradable drug-delivery system as described herein before, by either one or a combination of the following measures:
a) selecting the L/G ratio of the polylactic-co-glycolic acid (PLGA) units to adjust the hydrophobicity of the polymeric network;
b) selecting the L/G ratio of the polylactic-co-glycolic acid (PLGA) units to provide a sustained release of the active agent;
c) selecting the molar ratio of the amounts of the first to second crosslinkable precursors to adjust the hydrophobicity of the polymeric network; (=combining hydrophobic and hydrophilic precursors in varying ratios)
d) selecting the molar ratio of the amounts of the first to second crosslinkable precursors to provide a sustained release of the active agent;
e) selecting the type of hydrophobic liquid to be included, e.g., immobilized, in the organogel;
f) adding a third crosslinkable precursor that is less hydrolysable than the first and second, varying molar ratios of components;
g) dispersing an active agent that has high water solubility in particulate form into the hydrophobic phase;
h) incorporating degradable end groups onto the PLGA precursors to speed up the hydrolysis of the crosslinks relative to the PLGA internal ester linkages.

In the embodiments of the present invention involving PLGA units in the gel matrix, still another mechanism can be utilized to influence or control the release of the active agent. The hydrophobic properties of the covalently crosslinked polymeric network in the body of a patient may be varied by adjusting the ratio of lactic acid to glycolic acid units. By changing or selecting the L/G ratio of the polylactic-co-glycolic acid (PLGA) units, the hydrophobicity of the polymeric network can be altered. More hydrophobic lactic acid (L) units will increase the hydrophobicity of the gel matrix, and reduce swelling and water uptake; increasing the content of relatively more hydrophilic glycolic acid (G) units will decrease the hydrophobicity of the gel matrix, and will increase swelling and water uptake of the organogel.

Another possibility to adjust the hydrophobicity of the polymeric network is offered in embodiments of the invention by varying and/or selecting the molar ratio of the first to second crosslinkable precursors. Using hydrophobic precursors in a higher amount combined with more hydrophilic precursors such as PEG units, and vice versa, allow adjusting the swelling and hydrophobic liquid and/or active agent release.

Adding a third crosslinkable precursor that is different in its hydrophobicity than the first and second precursors and varying the molar ratios of the components can be further used to influence the swelling and hydrophobic liquid and/or active agent release, and the diffusion rates of active agent, hydrophobic liquid and/or water.

### Kit

In one aspect the invention further relates to kit comprising one or more sustained release biodegradable drug-delivery systems as described herein. The kit may further include instructions for using the system. In some embodiments, the kit comprises the parts of the drug-delivery system distributed over more than one separate containers for forming an organogel and/or an implant in-situ at a site of application or treatment site.

Kits for making the drug-delivery system of the invention may include premixed precursors and other components required for forming the organogel in separate compartments and applicators for combining the premixes and forming the organogel when needed, so that the precursors of the organogel are stored in the kit and made into the organogel/drug delivery system when needed for use with a patient. And kits may be made for applying an organogel as such, i.e., already in an organogel form. Applicators may be used in combination with the organogel. The kits are manufactured using medically acceptable conditions and contain components that have sterility, purity and preparation that is pharmaceutically acceptable. The kit may contain an applicator as appropriate, as well as instructions for use. The organogel components may be provided as: one or more containers of individual components or precursors, optionally premixed with the hydrophobic organic liquid and/or the active agent. Solvents/solutions may be provided in the kit or separately, or the components may be pre-mixed with the solvent. The kit may include syringes and/or needles for mixing and/or delivery. The kit or system may comprise the components set forth herein.

Packaging for a precursor and/or for an entire kit may be performed under dry conditions that are oxygen-free. The precursors and/or kit components may be placed in a hermetically sealed container that is not permeable to moisture or oxygen, for instance, glass or metal (foil) containers.

The organogels or premixes for making them may be gamma sterilized at the end of the implantable material manufacturing process. Alternatively, or furthermore, there may be a sterilization process either before and/or after assembly and sealing of a kit. Low moisture conditions may be used in this technique.

### Preferred Specific Embodiments

According to a specific embodiment, the sustained release, biodegradable drug-delivery system of the present invention is formed by gelling equimolar amounts of 4a20k PEG SAZ (20,000 Da PEG with 4 arms terminated with a succinimidyl azelate group) and 4a18k T1307 NH2 (18,000 Da Tetronic® 1307 with 4 arms terminated with amine groups) in the presence of 30 wt. % (based on the total dry weight of the system) acetyl triethyl citrate (ATEC) as the hydrophobic oil, 14 wt. % bupivacaine base (based on the total dry weight of the system) and dimethyl carbonate (DMC) as a solvent, and subsequently removing the solvent under reduced pressure.

According to another specific embodiment, the sustained release, biodegradable drug-delivery system of the present invention is formed by gelling in the presence of DMC:Acetone (90:10 w/w) identical amounts by weight of 4a18k-Tet1307-SAP-NHS as an electrophile functionalized precursor, (Tet1307 or T1307 is a 4 arm ethylenediamine tetrakis(ethoxylate-block-propoxylate)tetrol copolymer), and 4a20k PEG-NH2 as a nucleophile functionalized polyethylene glycol precursor. Tocopherol (vitamin E acetate) is used as the hydrophobic organic liquid in an amount of 42 wt.-% based on the dry gel, and 16 wt. % of micronized ropivacaine base (RPV) as the active agent, and the solvent is subsequently removed.

According to another specific embodiment, the sustained release, biodegradable drug-delivery system of the present invention is formed by gelling in the presence of DMC:Acetone (80:20 w/w) of 4a20k-PLGA-NHS as an electrophile functionalized polymeric precursor, crosslinked with a small molecule crosslinker TAEA in an amount of about 2.5 wt. % (based on the dry gel) using acetyl triethyl citrate (ATEC) oil as the hydrophobic organic liquid in an amount of 40 wt. % based on the dry gel, and 20 wt.-% based on the dry gel of micronized ropivacaine base (RPV) as the active agent, and subsequently removing the solvent.

According to another specific embodiment, the sustained release, biodegradable drug-delivery system of the present invention is formed by gelling in the presence of DMC:Acetone (80:20 w/w) same amounts of 4a20k-PLGA-NHS as an electrophile functionalized polymeric precursor, crosslinked with 4a18k Tet1307-NH2 as a nucleophile functionalized ethoxylate-block-propoxylate polymeric precursor, using acetyl triethyl citrate (ATEC) oil as the hydrophobic organic liquid in an amount of 29 wt. % based on the dry gel, and 14 wt.-% based on the dry gel of micronized ropivacaine base (RPV) as the active agent, and subsequently removing the solvent.

According to another specific embodiment, the sustained release, biodegradable drug-delivery system of the present invention is formed by gelling in the presence of DMC:Acetone (80:20 w/w) same amounts of 4a20k-Tet1307-SAP-NHS as an electrophile functionalized ethoxylate-block-propoxylate polymeric precursor, crosslinked with 4a3.6kTet701-NH2 as a nucleophile functionalized polymeric precursor, using acetyl triethyl citrate (ATEC) oil as the hydrophobic organic liquid in an amount of 37 wt. % based on the dry gel, and 19 wt.-% based on the dry gel of micronized ropivacaine base (RPV) as the active agent, and subsequently removing the solvent.

According to another specific embodiment, the sustained release, biodegradable drug-delivery system of the present invention is formed by gelling in the presence of DMC:Acetone (80:20 w/w) of 4a20k-PLGA-NHS as an electrophile functionalized polymeric precursor in an amount of about 40 wt. % (based on the dry gel), crosslinked with a small molecule crosslinker TAEA in an amount of about 0.4 wt. % (based on the dry gel), using acetyl triethyl citrate (ATEC) oil as the hydrophobic organic liquid in an amount of 40 wt. % based on the dry gel, and 20 wt.-% based on the dry gel of bupivacaine-HCl (BPV-HCl) as the active agent, and subsequently removing the solvent.

According to another specific embodiment, the sustained release, biodegradable drug-delivery system of the present invention is formed by gelling in the presence of DMC:Acetone (80:20 w/w) same amounts of 4a20k-PLGA-NHS as an electrophile functionalized polymeric precursor, crosslinked with 4a18k Tet1307-NH2 as a nucleophile functionalized ethoxylate-block-propoxylate polymeric precursor, using acetyl triethyl citrate (ATEC) oil as the hydrophobic organic liquid in an amount of 29 wt. % based on the dry gel, and 14 wt.-% based on the dry gel of bupivacaine-HCl (BPV-HCl) as the active agent, and subsequently removing the solvent.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Materials and Abbreviations Used in the Examples:

4a18kTet1307-SAP or 4a18kTet1307-SAP-NHS is a four arm 18 kDalton electrophile functionalized ethoxylate-block-propoxylate polymeric precursor (ethylenediamine tetrakis(ethoxylate-block-propoxylate)tetrol copolymer) obtained by functionalization of commercially available Tetronic 1307 with succinimidyl adipate (i.e., adipic acid and N-hydroxy succinimide (NHS)). 4a20k-Tet1307-SAP-NHS is the same precursor with a molecular weight of 20 kDalton.

4a18kTet1307-NH2 is a four arm 18 kDalton nucleophile functionalized ethoxylate-block-propoxylate polymeric precursor obtained by functionalization of commercially available Tetronic 1307 with hydroxylamine. 4a3.6kTet701-NH2 or 4a3.6kT701-NH2 is a four arm 3.6 kDalton nucleophile functionalized ethoxylate-block-propoxylate polymeric precursor obtained by functionalization of commercially available Tetronic 701 with hydroxylamine

4a20kSAZ or 4a20kPEG-SAZ or 4a20kPEG-SAZ-NHS is a four arm 20 kDalton electrophile functionalized polyethylene glycol precursor obtained by functionalization of commercially available 4a20kPEG with succinimidyl azelate (i.e., azelaic acid and N-hydroxy succinimide (NHS)).

4a20kNH2 or 4a20kPEG-NH2 is a nucleophile (amine) functionalized polyethylene glycol precursor having a molecular weight of 20 kDalton.

4a20kPLGA-NHS is a four arm 20 kDalton electrophile functionalized polymeric precursor obtained by functionalization of commercially available 4a20kPLGA (having an L/G ratio of 50:50) with N-hydroxy succinimide (NHS).

ATEC is acetyltriethyl citrate (triethyl 2-acetylcitrate), which is commercially available from Sigma-Aldrich/Merck

ATBC is acetyl tributyl citrate (tributyl O-acetylcitrate) which is commercially available from Sigma-Aldrich/Merck.

TAEA is tris(2-aminoethyl)amine, which is commercially available from Sigma-Aldrich/Merck.

DMC is dimethyl carbonate.

PBS is phosphate buffered saline of physiological salt concentration, pH7.4.

### Example 1

An organogel drug-delivery system has been produced in Example 1A using tocopherol (vitamin E acetate) as the hydrophobic organic liquid, micronized ropivacaine base (RPV) as the active agent, and two polymeric precursors, 4a18k-Tet1307-SAP-NHS as an electrophile functionalized precursor, (Tet1307 or T1307 is a 4 arm ethylenediamine tetrakis(ethoxylate-block-propoxylate)tetrol copolymer) and 4a20k PEG-NH2 as a nucleophile functionalized polyethylene glycol precursor. The Comparative Example 1B is without the hydrophobic organic liquid. The composition details are shown in Table 1 below.

Precursors, active agent, and hydrophobic organic liquid, if used, were combined with a mixture of DMC:Acetone (90:10 w/w) into a reaction mixture and tube casted to form a gel that was dried overnight to remove the solvents.

TABLE 1

| | Example 1A | | | Example 1B (comparative) | | |
|---|---|---|---|---|---|---|
| | | Amount | % w/w | | Amount | % w/w |
| Ingredients | | (mg) | dry | | (mg) | dry |
| Electrophile | 4a18k-Tet1307- | 250 | 21 | 4a18k-Tet1307- | 250 | 36 |
| | SAP-NHS | | | SAP-NHS | | |
| Nucleophile / | 4a20k-NH2 | 250 | 21 | 4a20k-NH2 | 250 | 36 |
| Crosslinker | | | | | | |
| Hydrophobic | Vit E acetate | 500 | 42 | — | — | |
| organic liquid | | | | | | |
| Active agent | RPV base | 187 | 16 | RPV base | 187 | 27 |
| | micronized | | | micronized | | |
| Solvent | DMC:Acetone | 500 | | DMC:Acetone | 500 | |
| | 90:10 w/w | | | 90:10 w/w | | |
| Total | | 1187 | 100 | | 687 | 100 |

Both compositions formed a solid gel in about 2 minutes of gelling time. While the comparative gel without oil component was transparent, the inventive gel of Example 1A was white opaque (cf. FIG. 2). No syneresis was observed in both gels.

### Example 2

Organogel drug-delivery systems have been produced using acetyl triethyl citrate (ATEC) oil as the hydrophobic organic liquid, micronized ropivacaine base (RPV) as the active agent, and 4a20k-PLGA-NHS as an electrophile functionalized polymeric precursor, crosslinked with a small molecule crosslinker TAEA in Example 2A (Table 2). The Comparative Example 2B is without the hydrophobic organic liquid.

Precursors, active agent, and hydrophobic organic liquid, if used, were combined with a mixture of DMC:Acetone (80:20 w/w) into a reaction mixture and tube casted to form a gel that was dried overnight to remove the solvents.

TABLE 2

| | Example 2A | | | Example 2B (comparative) | | |
|---|---|---|---|---|---|---|
| | | Amount | % w/w | | Amount | % w/w |
| Ingredients | | (mg) | dry | | (mg) | dry |
| Electrophile | 4a20k-PLGA- | 250 | 40 | 4a20k-PLGA- | 250 | 66 |
| | NHS | | | NHS | | |
| Nucleophile / | TAEA | 2.48 | 0.4 | TAEA | 2.48 | 0.7 |
| Crosslinker | | | | | | |
| Hydrophobic | ATEC oil | 250 | 40 | — | — | 0 |
| organic liquid | | | | | | |
| Active agent | RPV base | 125 | 20 | RPV base | 125 | 33 |
| | micronized | | | micronized | | |
| Solvent | DMC:Acetone | 500 | | DMC:Acetone | 500 | |
| | 80:20 w/w | | | 80:20 w/w | | |
| Total | | 627.48 | 100 | | 377.47 | 100 |

Both examples formed a solid gel in less than 3 minutes of gelling time. While the comparative gel without oil component was opaque and rigid, the inventive gel of Example 2A was opaque and soft and showed some syneresis after directly after producing, but no syneresis and a translucent appearance after drying.

An organogel drug-delivery system has been produced using acetyl triethyl citrate (ATEC) oil as the hydrophobic organic liquid, micronized ropivacaine base (RPV) as the active agent, and 4a20k-PLGA-NHS as an electrophile functionalized polymeric precursor, crosslinked with 4a18k Tet1307-NH2 as a nucleophile functionalized ethoxylate-block-propoxylate polymeric precursor in Example 2C (Table 3). The Comparative Example 2D is without the hydrophobic organic liquid.

TABLE 3

| | Example 2C | | | Example 2D (comparative) | | |
|---|---|---|---|---|---|---|
| | | Amount | % w/w | | Amount | % w/w |
| Ingredients | | (mg) | dry | | (mg) | dry |
| Electrophile | 4a20k-PLGA- | 250 | 29 | 4a20k-PLGA- | 250 | 40 |
| | NHS | | | NHS | | |
| Nucleophile / | 4a18kTet1307- | 250 | 29 | 4a18kTet1307- | 250 | 40 |
| Crosslinker | NH2 | | | NH2 | | |
| Hydrophobic | ATEC oil | 250 | 29 | — | — | 0 |
| organic liquid | | | | | | |
| Active agent | RPV base | 125 | 14 | RPV base | 125 | 20 |
| | micronized | | | micronized | | |
| Solvent | DMC:Acetone | 500 | | DMC:Acetone | 500 | |
| | 80:20 w/w | | | 80:20 w/w | | |
| Total | | 875 | 100 | | 625 | 100 |

Both examples formed a solid gel in less than 3 minutes of gelling time. Both gels had an opaque appearance and were rubbery and stretchable. No syneresis was observed in both gels.

A further organogel drug-delivery systems has been produced using acetyl triethyl citrate (ATEC) oil as the hydrophobic organic liquid, micronized ropivacaine base (RPV) as the active agent, and 4a20k-Tet1307-SAP-NHS as an electrophile functionalized ethoxylate-block-propoxylate polymeric precursor, crosslinked with 4a3.6kTet701-NH2 as a nucleophile functionalized polymeric precursor in examples in Example 2E (Table 4). The Comparative Example 2F is without the hydrophobic organic liquid.

TABLE 4

| | Example 2E | | | Example 2F (comparative) | | |
|---|---|---|---|---|---|---|
| | | Amount | % w/w | | Amount | % w/w |
| Ingredients | | (mg) | dry | | (mg) | dry |
| Electrophile | 4a20k-Tet1307- | 250 | 37 | 4a20k-Tet1307- | 250 | 59 |
| | SAP-NHS | | | SAP-NHS | | |
| Nucleophile / | 4a3.6kTet701- | 50 | 7 | 4a3.6kTet701- | 50 | 12 |
| Crosslinker | NH2 | | | NH2 | | |
| Hydrophobic | ATEC oil | 250 | 37 | — | — | 0 |
| organic liquid | | | | | | |
| Active agent | RPV base | 125 | 19 | RPV base | 125 | 29 |
| | micronized | | | micronized | | |
| Solvent | DMC:Acetone | 500 | | DMC:Acetone | 500 | |
| | 80:20 w/w | | | 80:20 w/w | | |
| Total | | 675 | 100 | | 425 | 100 |

Both examples formed a solid gel in more than 2 hours of gelling time. Both gels had an opaque appearance and where brittle and tacky. No syneresis was observed in both gels.

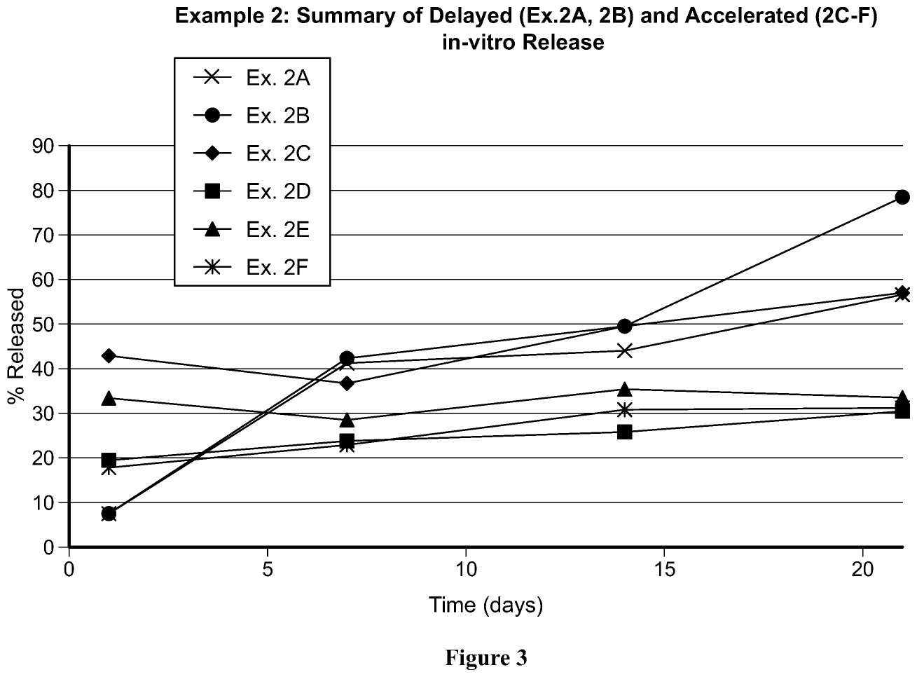

The release data of the inventive Example 2A (PLGA/TAEA gel) shows a delay of the ropivacaine release when compared to the comparative Example 2B without the organic hydrophobic liquid. The release data of the inventive Examples 2C and 2E (PLGA/Tet1307 or Tet1307/Tet701 gels) having more hydrophobic gelators each show an increase of the ropivacaine release when compared to the comparative Examples 2D and 2F without the organic hydrophobic liquid. The presence of an oil component in the organogel can be used to vary the release of an active agent that is more water than oil soluble, depending on the properties of the gel polymers used (cf. FIG. 3).

### Example 3

Organogel drug-delivery systems have been produced using acetyl triethyl citrate (ATEC) oil as the hydrophobic organic liquid, non-micronized Bupivacaine-HCl (BPV-HCl) as the active agent, and 4a20k-PLGA-NHS as an electrophile functionalized polymeric precursor, crosslinked with a small molecule crosslinker TAEA in Example 3A (Table 5). The Comparative Example 3B is without the hydrophobic organic liquid.

Precursors, active agent, and hydrophobic organic liquid, if used, were combined with a mixture of DMC:Acetone (80:20 w/w) into a reaction mixture and tube casted to form a gel that was dried overnight to remove the solvents.

TABLE 5

| | Example 3A | | | Example 3B (comparative) | | |
|---|---|---|---|---|---|---|
| | | Amount | % w/w | | Amount | % w/w |
| Ingredients | | (mg) | dry | | (mg) | dry |
| Electrophile | 4a20k-PLGA- | 250 | 40 | 4a20k-PLGA- | 250 | 66 |
| | NHS | | | NHS | | |
| Nucleophile / | TAEA | 2.48 | 0.4 | TAEA | 2.48 | 0.7 |
| Crosslinker | | | | | | |
| Hydrophobic | ATEC oil | 250 | 40 | — | — | 0 |
| organic liquid | | | | | | |
| Active agent | BPV-HCl | 125 | 20 | BPV-HCl | 125 | 33 |
| Solvent | DMC:Acetone | 500 | | DMC:Acetone | 500 | |
| | 80:20 w/w | | | 80:20 w/w | | |
| Total | | 627.48 | 100 | | 377.48 | 100 |

Both examples formed a solid gel in less than 3 minutes of gelling time. The gels were opaque and flexible and showed no syneresis.

An organogel drug-delivery system has been produced using acetyl triethyl citrate (ATEC) oil as the hydrophobic organic liquid, non-micronized Bupivacaine-HCl (BPV-HCl) as the active agent, and 4a20k-PLGA-NHS as an electrophile functionalized polymeric precursor, crosslinked with 4a18k Tet1307-NH2 as a nucleophile functionalized ethoxylate-block-propoxylate polymeric precursor in Example 3C (Table 6). The Comparative Example 3D is without the hydrophobic organic liquid.

TABLE 6

| | Example 3C | | | Example 3D (comparative) | | |
|---|---|---|---|---|---|---|
| | | Amount | % w/w | | Amount | % w/w |
| Ingredients | | (mg) | dry | | (mg) | dry |
| Electrophile | 4a20k-PLGA- | 250 | 29 | 4a20k-PLGA- | 250 | 40 |
| | NHS | | | NHS | | |
| Nucleophile / | 4a18kTet1307- | 250 | 29 | 4a18kTet1307- | 250 | 40 |
| Crosslinker | NH2 | | | NH2 | | |
| Hydrophobic | ATEC oil | 250 | 29 | — | — | 0 |
| organic liquid | | | | | | |
| Active agent | BPV-HCl | 125 | 14 | BPV-HCl | 125 | 20 |
| Solvent | DMC:Acetone | 500 | | DMC:Acetone | 500 | |
| | 80:20 w/w | | | 80:20 w/w | | |
| Total | | 875 | 100 | | 625 | 100 |

Both examples formed a solid gel in less than 3 minutes of gelling time having excellent gel properties. No syneresis was observed in both gels.

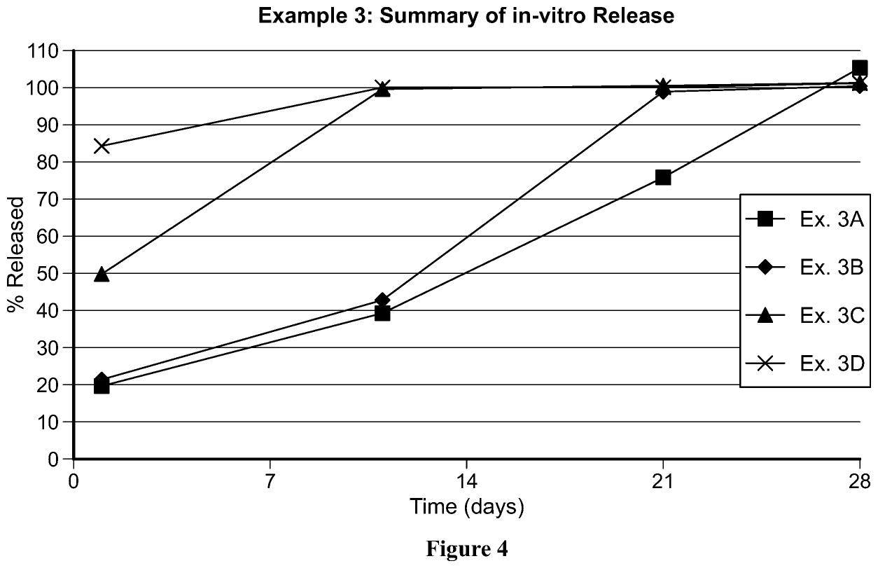

The in-vitro release kinetics (37° C., 1×PBS pH 7.4) of Examples 3A to 3D is shown in FIG. 4. As can be seen therein, the release of the active agent is delayed by the presence of the organic hydrophobic liquid, even though BPV is a more oil-soluble drug than Ropivacaine.

### Example 4

A series of organogel drug-delivery systems has been produced as described herein before using different oils as the hydrophobic organic liquid, non-micronized Bupivacaine-HCl (BPV-HCl) as the active agent, and 4a18k Tet1307-SAP-NHS (Table 8) or 4a20k-PLGA-NHS (Table 9) as an electrophile functionalized polymeric precursor, crosslinked with 4a18k Tet1307-NH2 as a nucleophile functionalized ethoxylate-block-propoxylate polymeric precursor. For comparison, each of the gels was also produced without the hydrophobic organic liquid.

The hydrophobic organic liquids used were acetyl triethyl citrate (ATEC), acetyl tributyl citrate (ATBC), α-tocopherol acetate (vitamin E acetate).

For preparing the organogels, two premixes were produced, the first premix including a mixture of the electrophile and hydrophobic organic liquid, if used, and 300 mg DMC:Acetone (80:20 w/w), the second premix including a mixture of the nucleophile, the active agent and 200 mg DMC:Acetone (80:20 w/w). Both premixes were combined into a reaction mixture and tube casted to form a gel that was dried overnight to remove the solvents. The compositions are detailed in Tables 7A, 7B, 8A and 8B below.

TABLE 7A

| Example 4A | | | Example 4B | | |
|---|---|---|---|---|---|
| | Amt. | % w/w | | Amt. | % w/w |
| Ingredients | (mg) | dry | Ingredients | (mg) | dry |
| Electrophile | 250 | 29 | Electrophile | 250 | 29 |
| 4a18k-Tet1307- | | | 4a18k-Tet1307- | | |
| SAP-NHS | | | SAP-NHS | | |
| Nucleophile | 250 | 29 | Nucleophile | 250 | 29 |
| 4a18k | | | 4a18k | | |
| Tet1307-NH2 | | | Tet1307-NH2 | | |
| Hydrophobic | 250 | 29 | Hydrophobic | 250 | 29 |
| organic liquid | | | organic liquid | | |
| ATEC oil | | | ATBC oil | | |
| Active agent | 125 | 14 | Active agent | 125 | 14 |
| BPV-HCl | | | BPV-HCl | | |
| Solvent | 500 | | Solvent | 500 | |
| DMC:Ac | | | DMC:Ac | | |
| 80:20 w/w | | | 80:20 w/w | | |
| Total | 875 | 100 | Total | 875 | 100 |

TABLE 7B

| Example 4C | | | Example 4D (comparative) | | |
|---|---|---|---|---|---|
| | Amt. | % w/w | | Amt. | % w/w |
| Ingredients | (mg) | dry | Ingredients | (mg) | dry |
| Electrophile | 250 | 29 | Electrophile | 250 | 40 |
| 4a18k-Tet1307- | | | 4a18k-Tet1307- | | |
| SAP-NHS | | | SAP-NHS | | |
| Nucleophile | 250 | 29 | Nucleophile | 250 | 40 |
| 4a18k | | | 4a18k | | |
| Tet1307-NH2 | | | Tet1307-NH2 | | |
| Hydrophobic | 250 | 29 | Hydrophobic | — | 0 |
| organic liquid | | | organic liquid | | |
| Vit. E Acetate | | | none | | |
| Active agent | 125 | 14 | Active agent | 125 | 20 |
| BPV-HCl | | | BPV-HCl | | |
| Solvent | 500 | | Solvent | 500 | |
| DMC:Ac | | | DMC:Ac | | |
| 80:20 w/w | | | 80:20 w/w | | |
| Total | 875 | 100 | Total | 625 | 100 |

TABLE 8A

| Example 4E | | | Example 4F | | |
|---|---|---|---|---|---|
| | Amt. | % w/w | | Amt. | % w/w |
| Ingredients | (mg) | dry | Ingredients | (mg) | dry |
| Electrophile | 250 | 29 | Electrophile | 250 | 29 |
| 4a20k-PLGA- | | | 4a20k-PLGA- | | |
| NHS | | | NHS | | |
| Nucleophile | 250 | 29 | Nucleophile | 250 | 29 |
| 4a18k | | | 4a18k | | |
| Tet1307-NH2 | | | Tet1307-NH2 | | |
| Hydrophobic | 250 | 29 | Hydrophobic | 250 | 29 |
| organic liquid | | | organic liquid | | |
| ATEC oil | | | ATBC oil | | |
| Active agent | 125 | 14 | Active agent | 125 | 14 |
| BPV-HCl | | | BPV-HCl | | |
| Solvent | 500 | | Solvent | 500 | |
| DMC:Ac | | | DMC:Ac | | |
| 80:20 w/w | | | 80:20 w/w | | |
| Total | 875 | 100 | Total | 875 | 100 |

TABLE 8B

| Example 4G | | | Example 4H (comparative) | | |
|---|---|---|---|---|---|
| | Amt. | % w/w | | Amt. | % w/w |
| Ingredients | (mg) | dry | Ingredients | (mg) | dry |
| Electrophile | 250 | 29 | Electrophile | 250 | 40 |
| 4a20k-PLGA- | | | 4a20k-PLGA- | | |
| NHS | | | NHS | | |
| Nucleophile | 250 | 29 | Nucleophile | 250 | 40 |
| 4a18k | | | 4a18k | | |
| Tet1307-NH2 | | | Tet1307-NH2 | | |
| Hydrophobic | 250 | 29 | Hydrophobic | — | 0 |
| organic liquid | | | organic liquid | | |
| Vit. E Acetate | | | none | | |
| Active agent | 125 | 14 | Active agent | 125 | 20 |
| BPV-HCl | | | BPV-HCl | | |
| Solvent | 500 | | Solvent | 500 | |
| DMC:Ac | | | DMC:Ac | | |
| 80:20 w/w | | | 80:20 w/w | | |
| Total | 875 | 100 | Total | 625 | 100 |

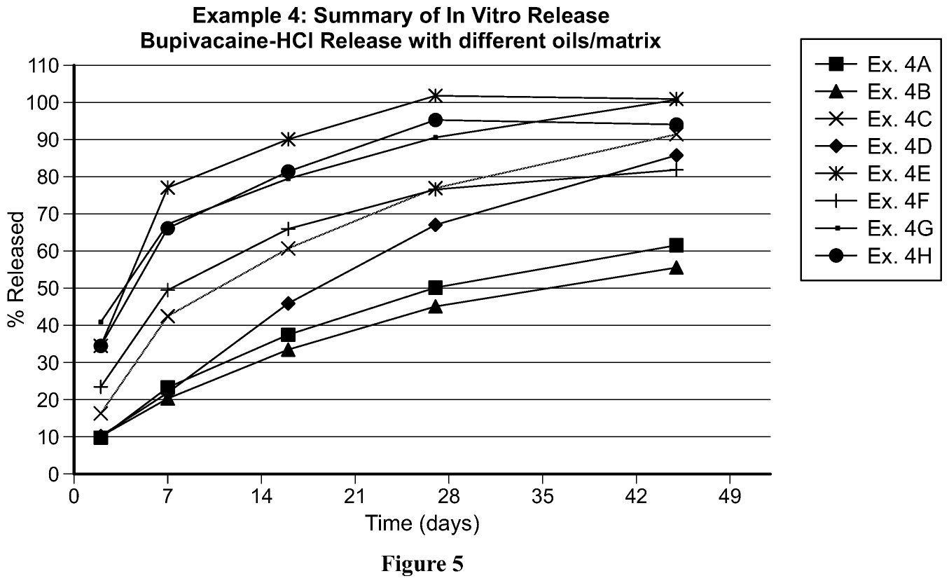

The in-vitro release kinetics (37° C., 1×PBS pH 7.4) of Examples 4A to 4H is shown in FIG. 5. As can be seen therein, the release of the active agent BPV-HCl from the Tetronic gels 4A to 4D is delayed by the presence of ATEC and ATBC, and accelerated by vitamin E acetate, each as compared with the no oil gel. For the PLGA gels 4E to 4H, the release of the active agent BPV-HCl is accelerated by ATEC and delayed by the presence of and ATBC and vitamin E acetate.

### Example 5

A series of organogel drug-delivery systems has been produced as described herein before using bupivacaine base (BPV Base) as the active agent, and three different polymer precursor formulations A, B and C as shown in Table 9. For each of the formulations, the hydrophobic organic liquids used were acetyl triethyl citrate (ATEC), or acetyl tributyl citrate (ATBC) in an amount of 0% (comparative) 20%, or 40% by weight of the formulation.

Formulations A include a hydrophilic polymer network (PEG-based) and were assigned a hydrophobicity value of 0% HB. Formulations B include a more hydrophobic polymer network (PEG-Poloxamer-based) and were assigned a hydrophobicity value of 15% HB. Formulations C include the most hydrophobic polymer network (PLGA-Poloxamer-based) and were assigned a hydrophobicity value of 65% HB.

TABLE 9

| | | | % Gelator | Active | | Oil |
|---|---|---|---|---|---|---|
| Formulation | Ester | Amine | hydrophobicity | agent | Oil | % (w/w) |
| A | 4a20kSAZ | 4a20kNH2 | 0 | BPV | ATEC | 0 |
| | | | | base | | 20 |
| | | | | | | 40 |
| | | | | | ATBC | 20 |
| | | | | | | 40 |
| B | 4a20kSAZ | 4a20kT1307NH2 | 15 | BPV | ATEC | 0 |
| | | | | base | | 20 |
| | | | | | | 40 |
| | | | | | ATBC | 20 |
| | | | | | | 40 |
| C | 4a20kPLGA | 4a20kT1307NH2 | 65 | BPV | ATEC | 0 |
| | 50:50 | | | base | | 20 |
| | | | | | | 40 |
| | | | | | ATBC | 20 |
| | | | | | | 40 |

All formulations have been produced as organogel strands with an average diameter of 2.9 mm and cut into pieces of 5 mm length, each having an average surface area of 59 mm², and an average drug load (dose) of 1600 μg.

The in-vitro release kinetics data (37° C., 1×PBS pH 7.4) of the formulations of Example 5 are shown in Table 10 below. As can be seen, the release of the active agent BPV Base from the PLGA gels of Formulations C do not persist 3 days but show a very small initial burst (2 h release value). Release is delayed by more hydrophobic oil ATBC as compared to ATEC and may be further delayed by improving crosslinking properties of the gels. For less hydrophobic gel Formulations B the burst data resembles that of the PLGA gels of Formulations C, but the gels delay the release of the BPV base longer, for up to about 5 days. A similar influence of the oil hydrophobicity is seen. Higher oil loading increases burst and the release per day initially. For the least hydrophobic gel Formulations A, higher oil amounts increase burst, but the release per day appears rather unaffected by the amount of oil. Overall, for delaying the BPV base release the Formulations B provide a good balance of properties. Samples having 20% (w/w) of oil overall gave the most reliable results largely avoiding initial burst.

TABLE 10

| | % BPV Base Released | | | | | |
|---|---|---|---|---|---|---|
| Formulation | 2 hr | Day 1 | Day 2 | Day 3 | Day 4 | Day 5 |
| A-0E | 32% | 52% | 77% | 92% | 94% | 87% |
| A-20E | 41% | 69% | 61% | 89% | 91% | 86% |
| A-40E | 39% | 74% | 82% | 86% | 84% | 88% |
| A-20B | 38% | 60% | 63% | 94% | 89% | 85% |
| A-40B | 53% | 59% | 63% | 75% | 90% | 89% |
| B-0E | 21% | 43% | 57% | 75% | 88% | 89% |
| B-20E | 36% | 48% | 65% | 61% | 82% | 91% |
| B-40E | 50% | 59% | 73% | 84% | 73% | 91% |
| B-20B | 22% | 45% | 64% | 75% | 95% | 90% |
| B-40B | 35% | 62% | 69% | 85% | 91% | 95% |
| C-0E | 28% | 36% | 61% | 77% | 100% | 100% |
| C-20E | 36% | 59% | 76% | 93% | 100% | 100% |
| C-40E | 26% | 61% | 60% | 72% | 100% | 100% |
| C-20B | 26% | 59% | 60% | 88% | 100% | 100% |

Nomenclature:
A-20E = Formulation A (4a20kPEG-SAZ/4a20kPEG-NH2) having 20% ATEC oil.
A-20B = Formulation A (4a20kPEG-SAZ/4a20kPEG-NH2) having 20% ATBC oil.

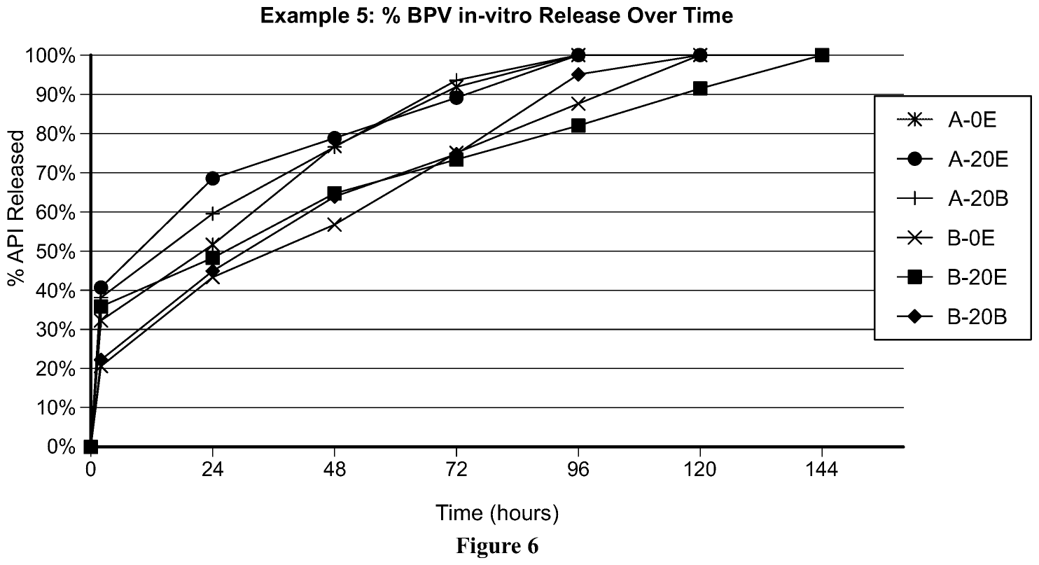

FIG. 6 illustrates the in vitro bupivacaine base release over time of several of the formulations A and B of Example 5.

### Example 6

Organogel drug-delivery systems have been produced using Travoprost as the active agent, which is simultaneously used as the hydrophobic organic liquid, and replaces it. Travoprost is a clear hydrophobic oil, which is practically insoluble in water. For the gel-forming components, hydrophobic 4a18K-Tet1307-SAP-NHIS was used as an electrophile functionalized polymeric precursor, crosslinked with 4a20K-NH2, a more hydrophilic amine (nucleophile) functionalized PEG. (Table 11).

Precursors, hydrophobic liquid active agent (travoprost) were combined with a mixture of DMC:Acetone (80:20 w/w) into a reaction mixture and tube casted to form a gel that was dried overnight to remove the solvents.

TABLE 11

Example 6

| | | Amount | % w/w |
|---|---|---|---|
| Ingredients | | (mg) | dry |
| Electrophile | 4a18K-Tet1307- | 250 | 25 |
| | SAP-NHS | | |
| Nucleophile/ | 4a20K-NH2 | 250 | 25 |
| Crosslinker | | | |
| Hydrophobic | Travoprost oil | 500 | 50 |
| organic liquid/ | | | |
| active agent | | | |
| Solvent | DMC:Acetone | 600 | |
| | 80:20 w/w | | |
| Total (dry) | | 1000 | 100 |

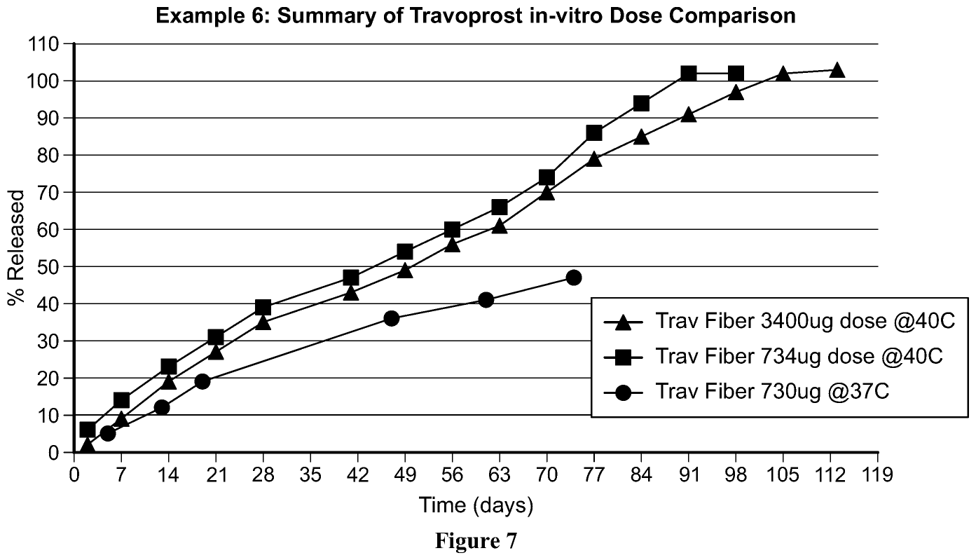

A solid gel was formed in less than 3 minutes of gelling time. The gels were opaque and flexible and showed no syneresis, and were cast into fibers. Pieces of the fiber corresponding to a drug dose of 3400 μg, 734 μg and 730 μg were cut and subjected to accelerated in-vitro release kinetics measurements at 40° C., 1×PBS pH 7.4 for the 3400 μg and 734 μg doses, and at regular body temperature conditions of 37° C., 1×PBS pH 7.4 for the 730 μg dose sample, each in 60 ml of buffer, so the 100% release corresponds to 4 times below sink conditions. The release experiment at 37° C. showed the expected slower release and was stopped after 6 weeks, an extrapolation of the curve shows that a continuous sustained release over approximately half a year is to be expected. The in-vitro release data is summarized in Table 12 below, and shown in FIG. 7.

TABLE 12

| | Example 6: % Travoprost released | | | |
|---|---|---|---|---|
| Time | Trav Fiber 3400 | Trav Fiber 734 | Time | Trav Fiber 730 |
| (days) | ug dose @40 C. | ug dose @40 C. | (days) | ug @37 C. |
| 2 | 2 | 6 | 5 | 5 |
| 7 | 9 | 14 | 13 | 12 |
| 14 | 19 | 23 | 19 | 19 |
| 21 | 27 | 31 | 47 | 36 |
| 28 | 35 | 39 | 61 | 41 |
| 41 | 43 | 47 | 74 | 47 |
| 49 | 49 | 54 | 49 | |
| 56 | 56 | 60 | 56 | |
| 63 | 61 | 66 | 63 | |
| 70 | 70 | 74 | 70 | |
| 77 | 79 | 86 | 77 | |
| 84 | 85 | 94 | 84 | |
| 91 | 91 | 102 | | |
| 98 | 97 | 102 | | |
| 105 | 102 | | | |
| 113 | 103 | | | |

As can be seen in FIG. 7, the active agent is released in a constantly slow sustained manner following zero order kinetics over an extended period of time. almost no burst is seen initially. With the gel expected to degrade substantially only after 6 months, the release kinetics of the active agent is diffusion controlled. Effects of degradation of the gel are not seen. Further, the comparison of two different doses shows that the analytical method was compatible and that the slow sustained release kinetics seen with the high dose sample was not due to drug saturation in the release buffer.

1-91. (canceled)

92. A sustained release, biodegradable drug-delivery system, comprising an organogel and an active agent, the organogel comprising:
a hydrophobic organic liquid, and
a biodegradable, covalently crosslinked polymeric network,
wherein the hydrophobic organic liquid and the active agent are contained in the biodegradable, covalently crosslinked polymeric network, and
wherein the biodegradable, covalently crosslinked polymeric network comprises a plurality of hydrophobic polymer units.

93. The system of claim 92, wherein the hydrophobic organic liquid is liquid at human body temperature.

94. The system of claim 92, wherein the active agent is dissolved or dispersed in the hydrophobic organic liquid.

95. The system of claim 92, wherein the hydrophobic organic liquid comprises an oil or oil mixture.

96. The system of claim 92, wherein the hydrophobic organic liquid is a biocompatible oil selected from triethyl citrate, acetyl triethyl citrate (ATEC), acetyl tributyl citrate (ATBC), α-tocopherol (vitamin E), α-tocopherol acetate; plant or vegetable oil, sesame oil, olive oil, soybean oil, sunflower oil, coconut oil, canola oil, rapeseed oil, nut oils, hazelnut, walnut, pecan, almond, cottonseed oil, corn oil, safflower oil, linseed oil, etc., ethyl oleate, castor oil, lipids being liquid at 37° C. or lower, saturated or unsaturated fatty acids, monoglycerides, diglycerides, triglycerides, isopropyl myristate, phospholipids, glycerophospholipids, sphingolipids, sterols, prenols, polyketides; hydrophobic biodegradable liquid polymers, PLGA, PGA, low melting point waxes, plant waxes, animal waxes, synthetic waxes, lanolin, jojoba oil, or combinations thereof.

97. The system of claim 92, wherein the biodegradable, covalently crosslinked polymeric network comprises one or more polymer units of polyethylene glycol, polyethylene oxide, polypropylene oxide, polyvinyl alcohol, poly(vinylpyrrolidinone), polylactic acid (PLA), polyglycolic acid (PGA), polylactic-co-glycolic acid (PLGA), p-dioxanone, trimethylene carbonate, caprolactone, random or block copolymers or combinations thereof, or one or more units of polyaminoacids, glycosaminoglycans, polysaccharides, or proteins.

98. The system of claim 97, wherein the covalently crosslinked polymeric network comprises a combination of:
a plurality of hydrophobic polymer units selected from at least one of polylactic acid (PLA), and polylactic-co-glycolic acid (PLGA), and
a plurality of at least one of hydrophilic polyethylene glycol (PEG) units, polypropylene glycol (PPG), or polyglycolic acid (PGA) units.

99. The system of claim 98, wherein the polymeric network comprises a combination of polylactic-co-glycolic acid (PLGA) units and polyethylene glycol (PEG) units.

100. The system of claim 99, wherein the ratio of polylactic-co-glycolic acid (PLGA) units to polyethylene glycol (PEG) units is about 2.5:1 to 1:2.5.

101. The system of claim 97, wherein the polylactic-co-glycolic acid (PLGA) units have an L/G ratio (in % L or G units) ranging from 0:100 to 100:0.

102. The system of claim 92, wherein the polymeric network is covalently crosslinked by hydrolysable bonds between polymer units.

103. The system of claim 92, wherein the polymeric network is formed from at least one covalently crosslinkable precursor that is miscible with the hydrophobic organic liquid, or soluble or dispersible in the hydrophobic organic liquid.

104. The system of claim 103, wherein the at least one crosslinkable precursor is a dendrimer or multi-arm precursor having a core and from 2 to 10 arms, each arm comprising a polymer unit and having a terminus.

105. The system of claim 103, wherein the at least one crosslinkable precursor comprises hydrophobic polymer units selected from polylactic acid (PLA) units and polylactic-co-glycolic acid (PLGA) units, copolymers or combinations thereof.

106. The system of claim 103, further including at least one crosslinker.

107. The system of claim 103, further comprising at least one crosslinkable precursor that is hydrophilic.

108. The system of claim 107, wherein the polymeric network comprises a covalently crosslinked combination of:
one or more multi-arm precursors selected from polyethylene glycol (PEG) and polyglycolic acid (PGA), and
one or more multi-arm precursors selected from polylactic acid (PLA), and polylactic-co-glycolic acid (PLGA).

109. The system of claim 92, wherein the active agent is selected from at least one of a therapeutically active agent or a diagnostically active agent or combinations thereof.

110. The system of claim 92, wherein the therapeutically active agent is selected from non-steroidal anti-inflammatory drugs (NSAIDS), steroids, antibiotics, pain relievers, analgesics, calcium-channel blockers, cell cycle inhibitors, chemotherapeutics, anti-viral drugs, anesthetics, hormones, anticancer drugs, antineoplastic agents, viruses, viruses for gene delivery, AAV, peptides, nanobodies, affibody molecules, ankyrins, DARPins, immunosuppressants, anti-inflammatory cytokine targeting agents, antiglaucoma agents, anti-VEGF agents, tyrosine kinase inhibitors, complement inhibitors, antihistamines, IL-6 inhibitors, HtRA1 inhibitors, RASP inhibitors, rho kinase inhibitors, plasma kallikrein inhibitors, nitric oxide donating PgAs, mast cell stabilizers, IGF-1 R inhibitors, TRPV1 antagonists, TrkA antagonists, pharmaceutically acceptable salts, anhydrates, hydrates, solvates, polymorphs, stereoisomers, crystalline forms, co-crystals, pro-drugs, conjugates, complexes and mixtures thereof.

111. A method of manufacturing a sustained release, biodegradable drug-delivery system, the method comprising the steps of:
(1) forming an organogel from at least:
a. a covalently crosslinked polymeric network,
b. a hydrophobic organic liquid, and
c. at least one active agent,
(2) wherein the hydrophobic organic liquid and the active agent are contained in the biodegradable, covalently crosslinked polymeric network,
(3) shaping the organogel.
